(19) United States
(12) Patent Application Publication
(10) Pub. No.: US 2004/0204486 A1
(43) Pub. Date: Oct. 14, 2004

(54) Anandamide and structurally related lipids as vanilloid receptor modulators

(76) Inventors: Edward Hogestatt, Lund (SE); Peter Zygmunt, Lund (SE)

Correspondence Address: FINNEGAN, HENDERSON, FARABOW, GARRETT & DUNNER, LLP, 1300 I STREET, NW, WASHINGTON, DC, 20005, US

(21) Appl. No.: 10/403,527

(22) Filed: Apr. 1, 2003

Related U.S. Application Data
(63) Continuation of application No. 09/849,972, filed on May 8, 2001, now abandoned.
Application No. 09/849,972 is a continuation-in-part of application No. 09/567,034, filed on May 8, 2000, now abandoned.

(51) Int. Cl. A61K 31/22; A61K 31/202

(52) U.S. Cl. 514/549; 514/560

(57) ABSTRACT

The invention discloses that anandamide is an endogenous ligand for vanilloid receptors, and especially the vanilloid receptor VR1. Other structurally related lipids, such as AM404, 1-arachidonylglycerol, and 2-arachidonylglycerol, are identified having vanilloid receptor activity as well. Methods of treating individuals suffering from, or at risk of suffering from, diseases and disorders associated with abnormal vanilloid receptor function are provided, as are methods of designing and identifying vanilloid receptor agonists and antagonists.

### CROSS-REFERENCE TO RELATED APPLICATION

This application is a continuation-in-part of U.S. application Ser. No. 09/567,034, filed May 8, 2000, priority to which is claimed, and the disclosure of which is hereby incorporated.

### BACKGROUND OF THE INVENTION

1. Field of the Invention

The present invention relates to the field of sensory nerve activation and signalling. More specifically, this invention relates to compounds and molecules that modulate the activity of vanilloid receptors, especially those on primary sensory nerves, and methods of identifying and using such compounds and molecules in the treatment of individuals.

2. Description of Related Art

The fatty acid amide anandamide (arachidonylethanolamide; see FIG. 1) was originally isolated from brain as an endogenous cannabinoid (CB) receptor ligand (ref. 1). Two pathways for biosynthesis of anandamide in neural tissues have been proposed. Anandamide might be formed either through phospholipase D-mediated hydrolysis of phospholipid N-arachidonyl phosphatidylethanolamine or through enzymatic condensation of arachidonic acid and ethanolamine (refs. 9, 10). According to the current view, the former pathway is predominantly or exclusively used, resulting in synthesis of anandamide and structurally related endogenous lipids, such as docosatetraenylethanolamide, di-homo-γ-linolenylethanolamide, and mead acid ethanolamide, primarily in the cell membrane (see ref. 31). The precursor phospholipid, N-acyl phosphatidylethanolamine, is produced by a trans-acylation reaction catalyzed by a calcium-dependent enzyme. Although this is currently the most widely held view on the mechanism of synthesis of these compounds, an enzymatic condensation of arachidonic acid and ethanolamine, catalyzed by anandamide synthase, might occur in some tissues.

Formation of anandamide has been demonstrated not only in nervous tissues, but in immunological cells and vascular endothelium as well (refs. 1, 11, 12). Indeed, macrophage-derived anandamide has recently been implicated in hemorrhagic shock (ref. 13) and endotoxin-induced hypotension (ref. 14). Thus, anandamide and structurally related lipids might be produced in a number of diseases and conditions involving activation of these biosynthetic pathways. Such potential diseases or conditions are, e.g., different allergic conditions; bronchial asthma; rhinitis; bladder hyper-reactivity; rheumatoid arthritis and other autoimmune diseases; atherosclerosis; cerebral vasospasm after subarachnoid hemorrhage; septic, hemorrhagic, and cardiac shock; gastroduodenal ulcers; infectious diseases; and different pain syndromes, including migraine and other forms of headache.

Anandamide is an agonist at both CB1 and CB2 receptors (refs. 5, 15, 16). CB1 receptors are expressed in the central and peripheral nervous system, whereas CB2 receptors are distributed mainly in cells of the immune system (ref. 5). Recently, mRNA transcripts encoding the CB1 receptor were detected in vascular endothelial and smooth muscle cells (refs. 11, 17). In anaesthetized rats, anandamide induces a prolonged vasodepressor response, which has been suggested to be mediated by pre-junctional inhibitory CB1 receptors on peripheral sympathetic nerve terminals (ref. 18). It is, however, unclear whether CB1 receptors are involved in the vasodilator effects of anandamide in isolated vascular preparations, which are deprived of their continuous sympathetic input (refs. 19, 20, 21, 22). Furthermore, binding of anandamide to neuronal receptors other than the CB receptors has not been reported.

More than a century ago, Högyes (1887) proposed that the pungent action of capsicol, an extract of Capsicum (hot peppers; chili peppers), was mediated by sensory nerves (see ref. 47). It was later shown that capsaicin, the active ingredient in hot peppers, and related vanilloid compounds (e.g., olvanil and resiniferatoxin) activate a subset of thin unmyclinated (C-fibers) and myelinated (Aδ-fibers) primary sensory nerves (refs. 47, 48). The existence on these nerves of a specific capsaicin recognition site, which was later termed the “vanilloid receptor”, was proposed by Szolcsanyi and Jancsó-Gábor (ref. 50) more than two decades ago.

Besides transmitting nociceptive information to the central nervous system and constituting the afferent limb of visceral reflexes (e.g., urogenital, respiratory, cardiovascular, and gastroenteric reflexes), capsaicin-sensitive primary sensory neurons also serve a local efferent function via release of neuropeptides from peripheral nerve endings (ref. 49). The efferent action of these nerves has profound effects on tissue function and homeostasis in many organs. In the vascular system, this mechanism is involved in neurogenic inflammation and metabolic/ischemic vasodilation. Via release of substance P and calcitonin gene-related peptide, these nerves may have both acute and trophic effects on the different cells in the vessel wall, including the vascular endothelium.

The vanilloid receptor (VR1), which was recently cloned by Caterina et al. (ref.

8), is a capsaicin-sensitive, heat-gated, non-selective cation channel. The work by Caterina et al. and subsequent studies have confirmed that VR1 is uniquely expressed in a subset of primary sensory neurons (ref 51), which are widely distributed in the human body and animals (see ref. 47). However, despite intense research, no endogenous ligand for this receptor has been described in the literature.

### SUMMARY OF THE INVENTION

The present invention is based on the discovery by the inventors that the non-vanilloid compound anandamide (arachidonylethanolamide) and structurally related lipids, such as N-(4-hydroxyphenyl)-5,8,11,14-eicosatetraenamide (AM404), 1-arachidonylglycerol, 2-arachidonylglycerol (FIG. 1), arachidonyl-3-methoxytyramine, arachidonyltyramine, docosatetraenylethanolamide, di-homo-γ-linolenylethanolamide, mead acid ethanolamide, methanandamide, and arachidonamide, modulate the activity of vanilloid receptors on primary sensory nerves. This discovery has numerous applications in the medical, pharmaceutical, and scientific fields, and provides a molecular mechanism for the non-CB1 receptor-mediated vasodilator action of anandamide.

In a first aspect, the invention provides methods of treating individuals (animals as well as humans). The methods of treatment include methods of treating diseases (including infections), disorders, and/or symptoms of diseases or disorders. In embodiments, the methods treat diseases, disorders, and/or symptoms that cause, or are otherwise associated with, abnormal activity of at least one vanilloid receptor. The methods of treating can be prophylactic, therapeutic, or curative. The methods can include administering anandamide or a structurally related lipid to an individual in an amount sufficient to bring about the intended treatment. In embodiments, the methods include administering a compound to an individual, wherein the compound affects the in vivo concentration of anandamide or a structurally related lipid. In embodiments, the methods include administering a compound to an individual, wherein the compound affects the in vivo ability of anandamide or a structurally related lipid to interact with, or otherwise affect the activity of, a vanilloid receptor.

Accordingly, this aspect of the invention provides for the use of anandamide or structurally related lipid compounds in the treatment of individuals. The anandamide or structurally related lipid compounds can be used to treat individuals suffering from a disease or disorder, or showing symptoms of a disease or disorder. Anandamide or structurally related compounds can be used prophylactically, therapeutically, or to cure a disease, disorder, or symptom.

In a second aspect, the invention provides methods of dilating or constricting vascular tissues, including, but not limited to, arteries, veins, and capillaries. In embodiments of this aspect of the invention, the methods include administering anandamide or a structurally related lipid to an individual in an amount that is sufficient to bring about dilation of at least one blood vessel. In other embodiments, the methods of this aspect of the invention include administering an inhibitor of anandamide or a structurally related lipid to an individual in an amount that is sufficient to bring about constriction of at least one blood vessel. An inhibitor is any compound or molecule that reduces the in vivo concentration of anandamide or a structurally related lipid, or reduces the ability of anandamide or a structurally related lipid to interact with a vanilloid receptor.

In a third aspect, the invention provides methods of modulating the activity of at least one vanilloid receptor. In embodiments, the methods of this aspect of the invention include administering anandamide or a structurally related lipid to an individual in an amount sufficient to activate at least one vanilloid receptor. In embodiments, the methods of this aspect of the invention include exposing a vanilloid receptor to anandamide or a structurally related lipid compound. In embodiments, the methods of this aspect of the invention include exposing a vanilloid receptor or anandamide (or a structurally related lipid) to a compound or molecule that inhibits interaction of the vanilloid receptor with the anandamide or related lipid. In embodiments, exposure results in physical contact between the receptor and the anandamide or structurally related lipid or between the receptor and the inhibitor. Accordingly, the methods of this aspect of the invention can be performed both in vivo and in vitro.

In another aspect, the invention provides methods of screening for individuals who are suffering from, or who are at risk for developing, a disease or disorder associated with abnormal vascular tone, inflammation, pain, or organ dysfunction. In embodiments, the methods include determining the in vivo concentration of anandamide or a structurally related lipid compound. The methods can further comprise comparing the concentration of anandamide or a structurally related lipid to a predetermined standard, or normal, concentration, and determining whether the concentration in the tested individual is below, above, or identical to the normal concentration. In embodiments, the methods of screening arepracticed in conjunction with the methods of treating provided by the invention.

In yet another aspect, the invention provides methods of diagnosing a disease or disorder. In embodiments, the methods of this aspect of the invention are methods of diagnosing the cause of a disease or disorder, wherein the cause is, or is related to, abnormal in vivo levels or activity of anandamide or a structurally related lipid. In embodiments, the methods of this aspect of the invention are methods of diagnosing the cause of a disease or disorder, wherein the cause is or is related to abnormal in vivo binding of anandamide or a structurally related lipid by a vanilloid receptor. The methods of this aspect of the invention can include determining the in vivo concentration of anandamide or a structurally related lipid. They can further comprise comparing the in vivo concentration of anandamide or a structurally related lipid to a pre-determined standard, or normal, concentration. In embodiments, the methods of diagnosing are practiced in conjunction with the methods of treating provided by this invention.

In a further aspect, the invention provides the ability to design analogs of anandamide, AM404, 1-arachidonylglycerol, and 2-arachidonylglycerol that affect vanilloid receptor function. Because the inventors have determined that anandamide and structurally related lipids, such as AM404, 1-arachidonylglycerol, and 2-arachidonylglycerol activate vanilloid receptors, and because the structure of these lipids are known, analogs can be rationally designed to provide beneficial attributes in addition to those of anandamide. Thus, in embodiments of this aspect of the invention, methods of designing analogs are provided. The methods can include identifying two- and three-dimensional structures that are important for interaction of such analogs with vanilloid receptors. The methods can include modifying portions of the anandamide, AM404, 1-arachidonylglycerol, and 2-arachidonylglycerol molecule other than the portions that are identified as important in binding to vanilloid receptors. In embodiments, the methods can include modifying the portions of anandamide, AM404, 1-arachidonylglycerol, and 2-arachidonylglycerol that are identified as being important in vanilloid receptor binding.

In yet another aspect, the invention provides methods of screening for compounds or molecules that interact with vanilloid receptors and modulate vanilloid receptor activity. Because the inventors have determined that anandamide, AM404, 1-arachidonylglycerol, and 2-arachidonylglycerol activate vanilloid receptors, and because their structures are known, identifying naturally occurring or synthetic compounds or molecules having the ability to bind and affect the activity of vanilloid receptors is now possible. In embodiments, the methods of screening comprise isolating or purifying the compound or molecule. The methods can include exposing at least one vanilloid receptor to a mixture of compounds and/or molecules, and isolating or purifying molecules that bind to, or otherwise affect the activity of, a vanilloid receptor. The methods can further comprise comparing at least one physical characteristic of the isolated or purified compound or molecule to anandamide. The methods can further comprise determining whether the compound or molecule has the ability to bind to and/or affect the activity of a vanilloid receptor. In embodiments, the vanilloid receptor is provided as a cloned receptor expressed on the surface of a recombinant cell. In embodiments, the method of screening is a high-throughput screening method. In embodiments, the methods are methods of screening for analogs of anandamide and structurally related lipids.

In yet a further aspect, the invention provides compositions comprising compounds or molecules that affect the activity of at least one vanilloid receptor. The compositions can comprise anandamide or a structurally related lipid compound. Alternatively, the compositions can comprise a compound or molecule that affects the activity of anandamide or a related lipid with respect to a vanilloid receptor. The compositions can include medicinal compounds intended for use in treating at least one disease, disorder, or at least one symptom of a disease or disorder. In embodiments, the compositions comprise anandamide or a structurally related lipid in an amount sufficient to bring about the desired result. For example, a composition of the invention can comprise anandamide in an aqueous or aqueous-organic solution, wherein the anandamide is present in a sufficient amount to bring about temporary dilation of arteries throughout the body of an individual to whom the composition is administered.

Accordingly, the invention provides kits containing compounds or molecules that affect the activity of at least one vanilloid receptor. The kits can contain anandamide or a structurally related lipid and/or compounds or molecules that affect the ability of anandamide or a structurally related lipid to bind to a vanilloid receptor. In embodiments, anandamide or a structurally related lipid is provided in the kit as the sole component of the kit. In embodiments, it is present as part of a composition. In embodiments, it is provided in combination with other compounds, solutions, or devices necessary or desirable for use of the compounds and/or compositions contained therein. Thus, the kits of the invention can contain all the necessary compounds, solutions, and equipment for administration of the compounds and compositions contained therein to an individual, or the kits can be designed for in vitro use of anandamide.

### BRIEF DESCRIPTION OF THE DRAWINGS

FIG. 1. Anandamide, AM404, 1-arachidonylglycerol, and 2-arachidonylglycerol are structurally related. These compounds consist of a polar head group containing at least one hydroxyl group, linked to arachidonic acid via an amide or ester bond.

FIG. 2. Effects of capsaicin and the CGRP receptor antagonist 8-37 CGRP on relaxations induce by anandamide (AEA) and acetylcholine (ACh) in rat and guinea pig arteries.

A. Traces showing typical relaxant responses to anandamide in these blood vessels contracted with phenylephrine (PhE; 1-3 μM) and prostaglandin F_{2α} (PG; 0.3-1 μM). Drug concentrations are given as -log molar concentrations. The horizontal scale marker represents 2 minutes and the vertical scale marker represents 2 mN. Dashed line indicates the basal tension level before addition of drugs. Each trace was obtained from different arterial segments.

B. Pre-treatment with capsaicin (10 μM) for 1 hour (followed by washout of capsaicin for 20 min) abolished the anandamide-induced relaxation, whereas subsequent application of acetylcholine (0.1-10 μM) always elicited a complete relaxation (mediated by endothelium-derived hyperpolarizing factor; refs. 19, 38, 43).

C. Concentration-response curves for anandamide after treatment with capsaicin (10 μM; ●), 8-37 CGRP (2 μM; ♦) for 30 minutes or vehicle (▴) in the different arteries (n=5-8).

FIG. 3. Release of CGRP from primary sensory nerves and effects of anandamide (AEA) and CGRP on cyclic AMP levels in the rat hepatic artery

A. The effect of 10 minute exposure to anandamide (10 μM) on CGRP contents was measured in the absence (n=11) and after either 20 minutes pre-incubation with 10 μM capsazepine (which was also present throughout the exposure to anandamide; n=5) or pre-treatment with capsaicin (10 μM) for 1 hour (followed by washout of capsaicin for 20 min; n=4). Control denotes preparations not exposed to anandamide (n=6). * P<0.05 compared controls, ^{#}P<0.05 compared to anandamide alone.

B. Cyclic AMP contents were measured after 5 minutes in the absence (control) or presence of either anandamide (AEA; 10 μM) or CGRP (10 nM) before and after pre-treatment with capsaicin (10 μM) for 1 hour (followed by washout of capsaicin for 20 min; n=6).

C. CGRP-like immunoreactive nerve fibers were observed in the adventitia. Bar=40 μm.

FIG. 4. Relaxant effects of endogenous and synthetic cannabinoid receptor agonists in rat hepatic and guinea pig basilar arteries.

A and B. Effects of the endocannabinoids 2-arachidonylglycerol (2-AG) and palmitylethanolamide (PEA), and arachidonic acid (AA) and ethanolamine (EA), which are breakdown products of anandamide, in (A) rat hepatic and (B) guinea pig basilar arteries contracted with phenylephrine (PhE; 1-3 μM) and prostaglandin F_{2α} (PG; 0.1-1 μM), respectively. As shown by traces, anandamide (AEA; 10 μM) or capsaicin (Cap; 10 μM) completely relaxed the vessels. Drug concentrations are given as -log molar concentrations. The horizontal scale marker represents 2 minutes and the vertical scale marker represents 2 mN. Dashed line indicates the basal tension level before addition of drugs. Each trace was obtained from different arterial segments.

C and D. Among the synthetic compounds tested (HU 210, WIN 55,212-2 and CP 55,940), only CP 55,940 caused a relaxation in (C) rat hepatic and (D) guinea pig basilar arteries. In contrast to anandamide, CP 55,940 also relaxed arteries after treatment with capsaicin (10 μM). As shown by traces, anandamide (10 μM) or capsaicin (10 μM) relaxed vessels exposed to HU210 and WIN 55,212-2. The number of experiments is shown within the brackets.

* P<0.05 compared to vehicle (ethanol 0.2%).

FIG. 5. Effect of the vanilloid receptor antagonist capsazepine on relaxations elicited by anandamide, methanandamide and capsaicin in rat hepatic, mesenteric and guinea pig basilar arteries.

A. Concentration-response curves for anandamide (n=5-6) and capsaicin (n=5-6) after incubation with capsazepine (3 μM) for 30 minutes (●) or vehicle (▴; 0.3% ethanol) in arteries contracted with phenylephrine (PhE; 1-3 μM; rat hepatic and mesenteric arteries) or prostaglandin F_{2α}. (PG; 0.1-1 μM; guinea pig basilar artery).

B and C. Concentration-response curves for capsaicin (n=6) and methanandamide (MethAEA; n=5-7) in the absence (∘) and presence of various concentrations of capsazepine (0.5 μM ▴; 0.8 μM ●; 1.0 μM ♦; 1.6 μM ▪; 3.2 μM ▾) in rat hepatic arteries.

D. Schild plots for capsazepine, using capsaicin (∘) or methanandamide (●) as agonists. For clarity, the individual data points are summarized as mean±s.e. mean (vertical lines; n=5-8). The slopes of the regression lines for capsaicin and methanandamide were (mean±SEM) 2.12±0.36 and 2.41±0.32, respectively (P>0.05).

FIG. 6. Concentration-dependent relaxations induced by AM404.

A. In the absence (control) and presence of the CGRP receptor antagonist 8-37 CGRP, and in preparations pre-treated with capsaicin.

B. In the absence (control) and presence of the vanilloid receptor antagonist capsazepine or the CB1 receptor antagonist SR141716A. Arteries were contracted by phenylephrine in the presence of Nω-nitro-L-arginine (0.3 mM) and indomethacin (10 μM). Data are presented as mean±S.E.M (n=5-7).

FIG. 7. Vanilloid receptor-dependent vasodilator action of different arachidonyl derivatives in rat isolated mesenteric arterial segments contracted with phenylephrine. Concentration response curves for (a) 1-arachidonylglycerol (1-AG) and (b) 2-arachidonylglycerol (2-AG) in the absence (●) and presence (▴) of the competitive vanilloid receptor antagonist capsazepine (1 μM). None of the agonists elicited a relaxation after pre-treatment with 10 μM capsaicin for 30 minutes (∘) to cause vanilloid receptor desensitization and/or depletion of sensory neuropeptides (ref. 54).

### DETAILED DESCRIPTION OF EMBODIMENTS

In a first aspect, the invention provides methods of treating individuals, including animals, such as pets (e.g., dogs, cats) and livestock (e.g., horses, cattle, pigs), and humans. The methods of treatment include methods of treating at least one disease, disorder, and/or symptom of at least one disease or disorder. The diseases and disorders, and the symptoms of diseases and disorders, include any medically recognized disease, disorder, or symptom. Examples include, but are not limited to, those caused by, or directly related to, 1) infections by viruses, bacteria, parasites, and fungi; 2) exposure to biological and non-biological environmental agents, such as ultra-violet light or other electromagnetic radiation, pollutants or other deleterious chemicals or compounds, pollen, and dust; 3) an individual's behavior or routine actions (e.g., hypertension, overeating, smoking tobacco products, etc.); and 4) a genetic predisposition to a disease or disorder. Examples of diseases, disorders, and symptoms include, but are not limited to, inflammation, pain, allergy or autoimmune disease, organ dysfunction, infection, and wounds.

In embodiments, the inflammation can be neurogenic inflammation, bronchial asthma, arthritis, inflammatory bowel disease, gout, allergic and vasomotor rhinitis, eczema, urticaria or hives, and/or psoriasis. In an exemplary embodiment, the method is a method of achieving reduction in inflammation, wherein the method comprises administering anandamide, AM404, or a structurally related lipid to an individual in an amount sufficient to achieve the desired amount of reduction in inflammation. The anandamide, AM404, or structurally related lipid can be administered, for example, by contact with skin or a mucous membrane, or by injection, locally, epidurally, or spinally.

In embodiments, the pain can be nociceptive pain, neurogenic pain, pain associated with anaesthesia, postherpetic neuralgia, pain associated with diabetic neuropathy, pain associated with chronic peripheral polyneuropathy, stump pain after amputation, postmastectomy pain syndrome, pain associated with arthritis (such as osteoarthritis), pain associated with benign and malignant tumors, pain associated with Gillain-Barrés disease, headache, and/or itching. In embodiments, the headache is migraine headache or Horton's headache. In an exemplary embodiment, the method is a method of achieving analgesia, wherein the method comprises administering anandamide, AM404, or a structurally related lipid to an individual in an amount sufficient to achieve analgesia. The anandamide, AM404, or structurally related lipid can be administered, for example, by contact with skin or a mucous membrane, or by injection, locally, epidurally, or spinally.

In embodiments, the allergy or autoimmune disease can be rheumatoid arthritis, rhinitis, conjunctivitis, and/or inflammatory bowel disease.

In embodiments, the organ dysfunction can be osteoarthritis, nasopharyngeal adenoids, bronchial asthma, atherosclerosis, urge incontinence or bladder hyper-reactivity, cough, gastroduodenal ulcer or other mucosal damage in the gastrointestinal tract, emesis, myocardial infarction, unstable angina, septic shock, hemorrhagic shock, cardiac shock, cerebral vasospasm after subarachnoid hemorrhage, stroke, and/or benign and malignant tumors.

In embodiments, the infection can be an infection by a bacterium, an infection by a virus, and/or an infection by a parasite. In embodiments, the infection is an infection by a herpesvirus.

Table 1 provides a list of non-exclusive, non-limiting applications provided by the methods of treatment according to the invention. Various symptoms, diseases, and disorders that are treatable according to the methods of the invention are listed.

TABLE 1
Indications for anandamide and structurally related lipids, including
AM404

Pain
Neurogenic Pain
Postherpetic neuralgia
Pain associated with diabetic neuropathy
Pain associated with chronic peripheral polyneuropathy
Stump pain after amputation
Postmastectomy pain syndrome
Pain associated with Gillain-Barres disease
Migraine
Horton's headache
Nociceptive Pain
Osteoarthritis
Arthritis
Gout
Anaesthesia
Epidural or spinal anesthesia
Local or regional anesthesia
Inflammatory Diseases
Allergic or vasomotor (non-allergic) rhinitis
Allergic or non-allergic conjunctivitis
Nasopharyngeal adenoids
Eczema
Bronchial asthma
Urticaria
Psoriasis
Inflammatory bowel disease
Atherosclerosis
Other Indications
Urge incontinence
Cough
Protection against ulcer and mucosal damage in the gastro-intestinal tract
Functional disorder of the gastro-intestinal tract
Emesis (nausea and vomiting)
Itching of various etiology
Wound healing
Herpes simplex infection
Myocardial infarction or unstable angina
Septic, hemorrhagic, and cardiac shock
Cerebral vasospasm
Stroke
Benign and malignant tumors

In embodiments, the methods treat at least one disease, disorder, and/or symptom that causes, or is otherwise associated with, abnormal activity of at least one vanilloid receptor. In preferred embodiments, the vanilloid receptor is a receptor known as VR1. In certain embodiments, the methods treat at least one disease, disorder, or symptom that causes, or is otherwise associated with, inactivation of at least one vanilloid receptor. Inactivation can be complete or incomplete. In certain embodiments, the methods treat at least one disease, disorder, or symptom that causes, or is otherwise associated with, hyperactivation of at least one vanilloid receptor. As used herein, hyperactivation of a vanilloid receptor means activation above a normal or average level seen in the relevant population as a whole.

The methods of treating can be prophylactic, therapeutic, or curative. When the methods of treating are practiced prior to an individual showing any clinical sign or symptom of a disease or disorder, they are considered prophylactic. Prophylactic treating can be practiced, for example, on individuals suspected of having a disease or disorder, or on individuals suspected of being at high risk of developing a disease or disorder. In embodiments, prophylactic methods reduce or eliminate the risk of developing a disease or disorder characterized by undesirable vasoconstriction. In embodiments, prophylactic methods reduce or eliminate the risk of developing a disease or disorder characterized by undesirable vasodilation. In embodiments, prophylactic methods reduce or eliminate the risk of developing a disease or disorder characterized by undesirable inflammation. In embodiments, prophylactic methods reduce or eliminate the risk of developing a disease or disorder characterized by undesirable pain. In embodiments, prophylactic methods reduce or eliminate the risk of developing a disease or disorder characterized by undesirable organ dysfunction. When the methods of treating are practiced on an individual already showing at least one clinical sign or symptom of a disease or disorder, the methods can be therapeutic or curative. Therapeutic methods are those methods that result in a detectable change in at least one symptom of the disease or disorder. Preferably, the detectable change is an improvement in the symptom. In embodiments, therapeutic methods reduce or eliminate undesirable vasoconstriction. In embodiments, therapeutic methods reduce or eliminate undesirable vasodilation. In embodiments, therapeutic methods reduce or eliminate undesirable inflammation. In embodiments, therapeutic methods reduce or eliminate undesirable pain. In embodiments, therapeutic methods reduce or eliminate undesirable organ dysfunction. Curative methods are those therapeutic methods that result in elimination of at least one symptom of a disease or disorder. Preferably, curative methods eliminate the cause of the disease or disorder. In embodiments, curative methods eliminate undesirable vasoconstriction. In embodiments, curative methods eliminate undesirable vasodilation. In embodiments, curative methods eliminate undesirable inflammation. In embodiments, curative methods eliminate undesirable pain. In embodiments, curative methods eliminate undesirable organ dysfunction.

In embodiments, the methods of treating include administering anandamide or a structurally related lipid to an individual in an amount sufficient to bring about the intended result. For example, in embodiments, anandamide or a structurally related lipid is administered in an amount sufficient to modulate vascular tone; in an amount sufficient to modulate inflammation; in an amount sufficient to modulate sensory nerve activity, in an amount sufficient to achieve analgesia, and/or in an amount sufficient to modulate organ function. In embodiments, anandamide, or a structurally related lipid, is administered to an individual in an amount sufficient to achieve a detectable change in the disease, disorder, or symptom being treated. The change can be a change throughout the body of the treated individual or at a specific site within or on the surface of the treated individual. Thus, the methods of treating include systemic treating as well as localized treating.

Reduction in vasoconstriction means any detectable increase in the diameter of blood vessels in the treated individual's body. Thus, the methods of treating by reduction in vasoconstriction are not limited to methods that dilate abnormally constricted blood vessels, but include reduction in vasoconstriction of blood vessels showing a normal or average amount of tone. Reduction in vasoconstriction can be detected in any number of ways known to those of skill in the art, including, but not limited to, detection of blood pressure, reduction of localized swelling or redness, and measurement of local blood flow with plethysmography or laser doppler. By corollary, in embodiments, the methods of treating include administering anandamide or a structurally related lipid in a sufficient amount to increase vasodilation a detectable amount throughout the body of the treated individual. In embodiments, anandamide, or a structurally related lipid, is administered to an individual in an amount sufficient to increase vasodilation a detectable amount at a specific site within, or on the surface of, a treated individual's body. Increasing vasodilation is not limited to blood vessels that are abnormally constricted, but instead includes dilation of any blood vessels found in any state of dilation or constriction.

Compounds that are “structurally related” to anandamide, AM404; 1-arachidonylglycerol, or 2-arachidonylglycerol can be represented by the following formula (I):

A-B-C (I)

in which A can be represented by

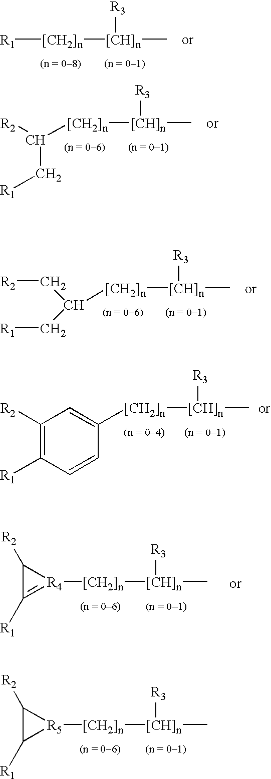

wherein R₁ can be any of the following substituents: —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br, and I, preferably hydroxy, methoxy, ethoxy, aminomethoxy, and aminoethoxy; and

wherein R₂ can be any of the following substituents: —H, —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br, and I, preferably hydroxy, methoxy, ethoxy, halon, and nitro;

wherein R₂ is not hydrogen when R₁ is alkoxy; and wherein any hydroxy group of R₁ and R₂ may be protected by a metabolically deprotectable protecting group to provide —OH in situ, wherein the metabolically deprotectable protecting group is a group comprising ester or amide characteristics, such as phenyl acetic acid derivatives and compounds such as those described in ref. 59; and

wherein R₃ can be —H, —CH₃, —C₂H₅, or —CF₃; and

wherein R₄ can be

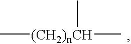

wherein n is 0-4; and

wherein R₅ can be

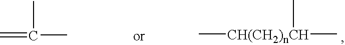

wherein n is 0-3; and—

in which B can be represented by —NHC(O)—, —NHC(S)—, —NHC(O)NH—, —NHS(O)—, —C(O)O—, —C(O)S—, —C(S)O—, —NHS—, —C(O)NH—, —C(S)NH—, —NHC(S)NH—, —S(O)NH—, —OC(O)—, —SC(O)—, —OC(S)— or —SNH—; and

in which C can be represented by an unsaturated straight or branched hydrocarbon chain, containing 6 to 24 carbon atoms, preferably 12 to 22 carbon atoms, and at least one double bond.

Compounds that are “structurally related” to anandamide, AM404, 1-arachidonylglycerol, or 2-arachidonylglycerol can also be represented by the following formula (II):

D-E-C (II)

in which D can be represented by

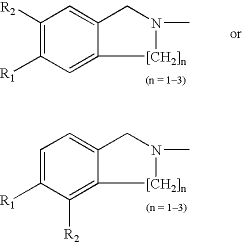

wherein R₁ can be any of the following substituents: —OH, —CH₂OH, —C₂H₅OH, —C₁₋₃-alkoxy, —CH₂OCH₃, —C₂H₅₀CH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br, and I, preferably hydroxy, methoxy, ethoxy, aminomethoxy and aminoethoxy; and

wherein R₂ can be any of the following substituents: —H, —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br, and I, preferably hydroxy, methoxy, ethoxy, halon, and nitro;

wherein R₂ is not hydrogen when R₁ is alkoxy; and wherein any hydroxy group of R₁ and R₂ may be protected by a metabolically deprotectable protecting group to provide —OH in situ, wherein the metabolically deprotectable protecting group is a group comprising ester or amide characteristics such as phenyl acetic acid derivatives, and those disclosed in ref. 59; and

in which E can be represented by —C(O)—, —C(S)—, —C(O)NH—, —C(S)NH—, —S(O)—, —S—, —O—, —C(O)O—, —C(O)S—, —OC(O)—, or C(S)O—; and

in which C can be represented by an unsaturated straight or branched hydrocarbon chain, containing 6 to 24 carbon atoms, preferably 12 to 22 carbon atoms, and at least one double bond.

The synthesis of these compounds can be carried out following known procedures, such as those described in refs. 57 and 58.

Examples of compounds that are structurally related to anandamide include, but are not limited to, AM404, 1-arachidonylglycerol, 2-arachidonylglycerol, arachadonamide, docosatetraenylethanolamide, di-homo-γ-linolenylethanolamide, mead acid ethanolamide, and acrylamides of monoamines or amino acids (such as arachidonyldopamine and arachidonyl-3-methoxytyramine arachidonylserine, arachidonylthreonine, arachidonyltyrosine).

In embodiments, the methods of treating include administering a compound or molecule to an individual, wherein the compound affects the in vivo concentration of anandamide or a structurally related lipid. The concentration of anandamide or a structurally related compound can be increased or decreased in response to administration of the compound or molecule. In embodiments, the concentration is affected by affecting the biosynthesis of anandamide or a structurally related lipid, for example by inhibiting or increasing the expression or activity of an enzyme required to produce anandamide, such as phosholipase D. In embodiments, the concentration is affected by increasing or decreasing the in vivo stability (half-life) of anandamide or a structurally related lipid. In embodiments, administration of the compound results in an increase in the concentration of anandamide or a structurally related lipid in the treated individual's body. In preferred embodiments, administration of the compound results in an increase in the concentration of anandamide or a structurally related lipid in nerve-containing tissues of the treated individual's body. In other embodiments, administration of the compound results in a decrease in the concentration of anandamide or a structurally related lipid in the treated individual's body. In embodiments, administration of the compound results in a decrease in the concentration of anandamide or a structurally related lipid in nerve-containing tissues of the treated individual's body. Preferably, the methods of treating result in a change in concentration of anandamide or a structurally related lipid at or in the immediate vicinity of at least one primary sensory neuron that expresses a vanilloid receptor. In preferred embodiments, the primary sensory neuron expresses a vanilloid receptor known as VR1.

In embodiments, the methods include administering a compound to an individual, wherein the compound affects the in vivo ability of anandamide or a structurally related lipid to interact with, or otherwise affect the activity of, a vanilloid receptor. In embodiments, the compound binds to anandamide or a structurally related lipid and blocks binding of the anandamide or related lipid to a vanilloid receptor. In embodiments, the compound binds to anandamide or a structurally related lipid and enhances binding of the anandamide or structurally related lipid to a vanilloid receptor.

Examples of compounds or molecules that affect the in vivo activity or concentration of anandamide or a structurally related lipid include, but are not limited to, inhibitors of fatty acid amidohydrolases, inhibitors of membrane transporters, inhibitors of phospholipase D, activators of phospholipase D, anandamide synthase, inhibitors of anandamide synthase, and activators of anandamide synthase, inhibitors of vanilloid receptors, and activators of vanilloid receptors.

The methods of treating can include administering at least one other compound or molecule to an individual. The other compound can, but is not necessarily, biologically active. Examples of other compounds or molecules include, but are not limited to, water or aqueous solutions, salts, atomic elements, such as calcium, phosphorous, potassium, iron, etc., and drugs, such as anti-inflammatories, antibiotics, and pain killers (including local anaesthetics). In general, any compound or molecule, or combination thereof, known in the art to be suitable for administration to an individual can be used. Preferably, the other compound(s) provides a beneficial effect to the treated individual, for example by treating the disease or disorder from which the individual suffers, or treating a symptom of the disease or disorder. Preferably, the other compound(s) does not reduce or eliminate the beneficial effects of the compound that is originally included to treat the disease, disorder, or symptom.

The methods of treating can include a single administration to an individual, or can include multiple administrations. Treatment and dosing regimens can be designed and implemented in accordance with those that are well-known and widely practiced in the art. It is contemplated that each regimen will be tailored to the individual to be treated and the disease(s), disorder(s), and/or symptom(s) involved. However, such individual tailoring is well within the skill of those in the art and does not involve undue or excessive experimentation.

In preferred embodiments, the method of treating is a method of treating pain. In other preferred embodiments, the method of treating is a method of treating inflammation. In yet other preferred embodiments, the method of treating is a method of treating organ dysfunction, such as bladder instability. The methods of these embodiments include administering anandamide or a structurally related lipid to an individual suffering from these disorders. The anandamide or structurally related lipid is administered in an amount sufficient to reduce or eliminate the disorder. For example, in embodiments where the method treats pain, the anandamide or structurally related lipid is administered in an amount sufficient to reduce or eliminate the pain. As another example, in embodiments where the method treats inflammation, the anandamide or structurally related lipid is administered in an amount sufficient to reduce or eliminate the inflammation. The amount administered can be determined by one of skill in the art without undue experimentation based on dosing regimens known and practiced in the art. As indicated above, the method of treating can reduce, but not eliminate, the disorder. This is particularly relevant where the methods are directed to treatment of pain. Reduction, but not elimination, of a specific pain is often desirable so that the individual can self-monitor other potential pain sites, or the progression of an associated disease or disorder. When the treatment is intended to reduce, but not eliminate, the pain, the amount of reduction in pain can be determined based on the symptoms shown by the patient and/or based on an underlying disease or disorder.

Accordingly, this aspect of the invention provides for the use of anandamide or structurally related lipid compounds in the treatment of individuals. The anandamide or structurally related lipid compounds can be used to treat individuals suffering from a disease or disorder, or showing symptoms of a disease or disorder. As discussed in detail above, anandamide or structurally related compounds can be used prophylactically, therapeutically, or to cure a disease, disorder, or symptom. In addition, this aspect of the invention provides for the use of compounds and molecules that affect the in vivo activity and/or concentration of anandamide or structurally related lipid, wherein the use results in treatment of individuals suffering from a disease or disorder, or showing symptoms of a disease or disorder.

In a second aspect, the invention provides methods of modulating vascular tone, inflammation, sensory nerve activity, nociception (i.e., pain perception), or organ function. The methods can be methods of dilating vascular tissues or methods of constricting vascular tissues, methods of decreasing tissue inflammation, or methods of decreasing nociception. The vascular tissues, include, but are not necessarily limited to, arteries, veins, and capillaries. In embodiments, the methods of modulating vascular tone, inflammation, nociception, or organ function include administering a compound or molecule that affects at least one cell of the central or the peripheral nervous system. In preferred embodiments, the methods include administering a compound or molecule that affects the activity of at least one cell of the peripheral nervous system. In preferred embodiments, the methods include modulating the activity of at least one primary sensory neuron.

In embodiments of this aspect of the invention, the methods include administering anandamide or a structurally related lipid to an individual in an amount that is sufficient to increase dilation or decrease constriction of at least one blood vessel. For example, anandamide can be administered to an individual in an amount sufficient to completely dilate a coronary or cerebral artery. In other embodiments, the methods of this aspect of the invention include administering a compound or molecule to an individual in an amount sufficient to reduce or inhibit the in vivo activity of anandamide or a structurally related lipid. In yet other embodiments, the methods of this aspect of the invention include administering a compound or molecule to an individual in an amount sufficient to reduce the in vivo concentration of anandamide or a structurally related lipid. For example, the methods can be used to increase systemic blood pressure of an individual suffering from, or at risk of, low blood pressure (e.g., hemorrhagic or septic shock). In these other embodiments, the methods result in vasoconstriction.

The methods of modulating vascular tone, inflammation, sensory nerve activity, nociception, or organ function can include administering at least one other compound or molecule to an individual. The other compound can, but is not necessarily, biologically active. Examples of other compounds or molecules include, but are not limited to, water or aqueous solutions, salts, atomic elements, such as calcium, phosphorous, potassium, iron, etc., and drugs, such as anti-inflammatories, antibiotics, and pain killers, including local anaesthetics. In general, any compound or molecule, or combination thereof, known in the art to be suitable for administration to an individual can be used. Preferably, the other compound(s) provides a beneficial effect to the treated individual, for example by treating a disease or disorder from which the individual suffers, or treating a symptom of the disease or disorder. Preferably, the other compound(s) does not reduce or eliminate the effects of the vasomodulatory compound.

The methods of modulating vascular tone, inflammation, sensory nerve activity, nociception, or organ function can include a single administration to an individual, or can include multiple administrations. Treatment and dosing regimens can be designed and implemented in accordance with those that are well-known and widely practiced in the art. It is contemplated that each regimen will be tailored to the individual to be treated and the desired outcome. Such individual tailoring is well within the skill of those in the art and does not involve undue or excessive experimentation.

Furthermore, the methods of modulating vascular tone, inflammation, sensory nerve activity, nociception, or organ function can include determining whether, and to what extent, vascular tone, inflammation, sensory nerve activity, nociception, or organ function was affected. Any known technique for detecting and/or quantifying vascular tone, inflammation, sensory nerve activity, nociception, or organ function, and/or changes in vascular tone, inflammation, sensory nerve activity, nociception, or organ function, can be used. Preferred techniques include, but are not limited to, detection of blood pressure, reduction of localized swelling or redness, measurement of local blood flow with plethysmography or laser doppler, and those disclosed in the Examples below.

Accordingly, this aspect of the invention provides for the use of anandamide or structurally related lipid compounds in the modulation of vascular tone, inflammation, sensory nerve activity, nociception, or organ function through modulation of the activity of vanilloid receptors. In addition, this aspect of the invention provides for the use of compounds and molecules that affect the in vivo activity and/or concentration of anandamide or structurally related lipids, wherein the use modulates vascular tone, inflammation, sensory nerve activity, nociception, or organ function.

In a third aspect, the invention provides methods of modulating the activity of at least one vanilloid receptor. In preferred embodiments, the vanilloid receptor is the receptor known as VR1. In certain embodiments of this aspect of the invention, the methods of modulating the activity are methods of activating at least one vanilloid receptor. In embodiments, the methods of this aspect of the invention include administering anandamide or a structurally related lipid to an individual in an amount sufficient to activate at least one vanilloid receptor. In other embodiments, the methods include administering a compound or molecule to an individual in an amount sufficient to increase the in vivo activity or concentration of anandamide or a structurally related compound. In embodiments, the methods of this aspect of the invention include exposing a vanilloid receptor to anandamide or a structurally related lipid compound. In preferred embodiments, exposure results in physical contact between the receptor and the anandamide or structurally related lipid.

In certain embodiments of this aspect of the invention, the methods of modulating the activity of at least one vanilloid receptor are methods of inhibiting the activity of the receptor(s). In embodiments, the methods of this aspect of the invention include administering a compound or molecule to an individual in an amount sufficient to bring about the desired result. In embodiments, the compound or molecule inhibits the activity of anandamide or a structurally related lipid. In embodiments, the compound or molecule reduces the ability of anandamide or a structurally related lipid to bind to at least one vanilloid receptor. In embodiments, the methods of this aspect of the invention include exposing a vanilloid receptor to anandamide or a structurally related lipid compound. In certain embodiments, exposure results in physical contact between the receptor and the anandamide or structurally related lipid.

Because the methods of this aspect of the invention can include contacting at least one vanilloid receptor with anandamide or a structurally related lipid, but do not require that the vanilloid receptor be present in vivo, this aspect of the invention can be performed both in vivo and in vitro. Furthermore, the methods of modulating the activity of at least one vanilloid receptor can include determining whether, and to what extent, the activity of the vanilloid receptor(s) was affected. Any known technique for detecting and/or quantifying the activity of vanilloid receptors can be used. Preferred techniques are disclosed in the Examples below. In addition to these examples, any modulation of vanilloid receptor activity can be detected in cells expressing native or cloned vanilloid receptors by use of the patch-clamp technique, calcium imaging, radioligand binding techniques, or calcium influx measurements (refs. 8, 11, 23, 24).

In another aspect, the invention provides methods of screening for individuals who are suffering from, or who are at risk for developing, a disease or disorder associated with abnormal vascular tone, inflammation sensory nerve activity, nociception, or organ function, abnormal levels of anandamide or a structurally related lipid, or abnormal activity of at least one vanilloid receptor. In preferred embodiments, the methods include determining the in vivo concentration of anandamide or a structurally related lipid compound. The methods can further comprise comparing the concentration of anandamide or a structurally related lipid to a pre-determined standard, or normal, concentration, and determining whether the concentration in the tested individual is below, above, or identical to the normal concentration. In preferred embodiments where an abnormal concentration of anandamide or a structurally related lipid is detected, the methods of screening are practiced in conjunction with the methods of treating provided by this invention.

Typically, if the concentration of anandamide or a structurally related lipid in the tested individual is determined to be above normal, excessive vasodilation, inflammation, pain, or organ dysfunction due to the activity of at least one vanilloid receptor is indicated, and a treatment regimen can be implemented based on this information. However, if the concentration in the tested individual is above normal, yet symptoms of excessive vasodilation, inflammation, pain, or organ dysfunction are not seen in the individual, or even symptoms of vasoconstriction are seen, in activity of at least one vanilloid receptor is indicated, and an appropriate treatment regimen can be implemented based on this information. Alternatively, if the concentration of anandamide or a structurally related lipid in the tested individual is determined to be normal or average for the relevant population, yet symptoms of excessive vasodilation, inflammation, pain, organ dysfunction, or vasoconstriction are seen in the tested individual hyperactivity or inactivity (respectively) of at least one vanilloid receptor is indicated. Based on this information, an appropriate treatment regimen can be implemented. Furthermore, if the concentration of anandamide or a structurally related lipid is determined to be below normal in the tested individual, vasoconstriction, inflammation, pain, or organ dysfunction due to the action of at least one vanilloid receptor is indicated. Accordingly, if symptoms consistent with vasoconstriction are seen, an appropriate treatment regimen can be implemented. Of course, if below normal concentrations of anandamide or a structurally related lipid are detected, and yet no symptoms of excessive vasoconstriction, inflammation, pain, or organ dysfunction are seen, or even excessive vasodilation is seen, hyperactivity of at least on vanilloid receptor is indicated.

The discovery that anandamide and structurally related lipids modulate vanilloid receptor activity permits one to screen for predispositions to a disease or disorder, or subclinical (e.g., incubation or developmental) states of a disease or disorder. That is, because the present invention provides a nexus between the in vivo concentration of anandamide or structurally related lipids, it is now possible to screen individuals who do not evince clinical or outward signs of a disease or disorder, but who, in fact, have or are at risk of developing a disease or disorder. Examples of diseases and disorders that can be screened for are listed in Table 1 above.

In yet another aspect, the invention provides methods of diagnosing a disease or disorder. In embodiments, the methods of this aspect of the invention are methods of diagnosing the cause of a disease or disorder, wherein the cause is, or is related to, abnormal in vivo levels or activity of anandamide or a structurally related lipid. In embodiments, the methods of this aspect of the invention are methods of diagnosing the cause of a disease or disorder, wherein the cause is, or is related to, abnormal in vivo binding of anandamide or a structurally related lipid to a vanilloid receptor. The methods of this aspect of the invention can include determining the in vivo concentration of anandamide or a structurally related lipid. They can further comprise comparing the in vivo concentration of anandamide or a structurally related lipid to a predetermined standard, or normal, concentration. In embodiments, the methods of diagnosing are practiced in conjunction with the methods of treating provided by this invention. Exemplary diseases and disorders that can be diagnosed are listed in Table 1 above.

In a further aspect, the invention provides the ability to develop and/or design agonists and antagonists of vanilloid receptors. In embodiments, this aspect of the invention enables one to design analogs of anandamide and structurally related lipids that affect vanilloid receptor function. Because the inventors have determined that anandamide and structurally related lipids activate vanilloid receptors, and because the structures of anandamide and the related lipids are known, analogs can be rationally designed to provide beneficial attributes similar to, or in addition to, those of anandamide and structurally related lipids. In particular, the agonists and antagonists can have structures represented by formulas (I) and (II) disclosed above.

Thus, in embodiments of this aspect of the invention, methods of designing analogs are provided. In preferred embodiments, the methods of designing analogs include designing and chemically synthesizing molecules that are structurally related to anandamide. In particular, the agonists and antagonists can have structures represented be formulas (I) and (II) disclosed above.

The methods can include identifying two- and three-dimensional structures that are important for interaction of anandamide and structurally related lipids with vanilloid receptors. The methods can further include modifying portions of the anandamide, AM404, 1-arachidonylglycerol, and 2-arachidonylglycerol molecules other than the portions that are identified as important in binding to vanilloid receptors. In embodiments, the methods can include modifying the portions of anandamide, AM404, 1-arachidonylglycerol, and 2-arachidonylglycerol that are identified as being important in vanilloid receptor binding. Analogs according to the invention can have higher vanilloid receptor binding activity than anandamide, lower vanilloid receptor binding activity than anandamide, or equivalent vanilloid receptor binding activity to anandamide. Furthermore, analogs according to the invention can have differing levels of vanilloid receptor binding activity relative to anandamide when assayed in vivo and in vitro. Accordingly, vanilloid receptor modulating activity of the analogs can be tested in vitro, in vivo, or both. Preferably, the analogs are tested for their activity on the vanilloid receptor known as VR1. Preferably, in vitro activity is tested using recombinant cells expressing cloned VR1.

In a preferred embodiment, the method is a method of developing agonists and antagonists of a vanilloid receptor, wherein the method comprises

a) obtaining a compound according to formula (I) or formula (II), and

b) testing the compound for its ability to modulate the activity of at least one vanilloid receptor,

wherein modulation of activity indicates that the tested compound is an agonist or antagonist of a vanilloid receptor.

In some preferred embodiments, the agonists and antagonists are obtained by chemical synthesis. In other preferred embodiments, the agonists and antagonists are obtained from biologically produced mixtures or compositions. In embodiments, the method is performed in vitro, for example by using cells expressing a recombinant VR1 receptor. The method is particularly applicable to high-throughput screening.

In yet another aspect, the invention provides methods of screening for compounds or molecules that interact with vanilloid receptors. Because the inventors have determined that anandamide and structurally related lipids activate vanilloid receptors, and because the structures of anandamide and these related lipids are known, identifying other naturally occurring or synthetic compounds or molecules having the ability to bind and affect the activity of vanilloid receptors is now possible.

In embodiments of this aspect of the invention, the methods of screening include exposing at least one vanilloid receptor to a mixture of compounds and/or molecules, and isolating or purifying molecules that bind to, or otherwise affect the activity of, a vanilloid receptor. As used herein, isolating and/or purifying means removing the active compound from at least one other compound or molecule (other than the solvent used) that is present in the starting mixture. Thus, isolating or purifying can result in partial purification of the active molecule, or complete purification and isolation of the molecule. Vanilloid receptor activity can be detected and quantitated using any suitable method. Preferred methods are disclosed in the Examples. In addition to these examples, any modulation of vanilloid receptor activity can be detected in cells expressing cloned vanilloid receptors using the patch-clamp technique, calcium imaging, radioligand binding techniques, or calcium influx measurements (refs. 8, 11, 23, 24).

The methods can further comprise comparing at least one physical characteristic of the isolated or purified compound or molecule to anandamide or a structurally related lipid. The methods can further comprise determining whether the compound or molecule has the ability to bind to and/or affect the activity of a vanilloid receptor. In embodiments, the vanilloid receptor is provided as a cloned receptor expressed on the surface of a recombinant cell. Preferably, the cloned receptor is that known as VR1. In embodiments, the method of screening is a high-throughput screening method. Methods for high-throughput screening are well known in the art, and thus need not be described in detail here. In embodiments, the methods are methods of screening for analogs of anandamide or a structurally related lipid.

In yet a further aspect, the invention provides compositions comprising at least one compound or molecule that affects the activity of a vanilloid receptor. In embodiments, the compositions comprise anandamide or a structurally related lipid compound. In embodiments, the compositions comprise a compound or molecule that affects the concentration or activity of anandamide or a structurally related compound. The compositions can be, but are not necessarily, medicinal compounds intended for use in treating at least one disease or disorder, or at least one symptom of a disease or disorder. In embodiments, the compositions comprise anandamide or a structurally related lipid in an amount sufficient to bring about the desired result. As examples, a composition of the invention can comprise anandamide or a structurally related lipid in an aqueous or aqueous-organic solution, wherein such a lipid is present in a sufficient amount to bring about temporary dilation of arteries, analgesia, an anti-inflammatory effect, or rectify organ dysfunction in an individual to whom the composition is administered. In embodiments, the compositions comprise a compound or molecule that reduces the concentration of anandamide or a structurally related lipid, or reduces the ability of anandamide or a structurally related lipid to affect the activity of a vanilloid receptor, wherein the compound or molecule is present in an amount sufficient to bring about a desired change in vanilloid receptor activity.

The compositions of the invention can comprise anandamide or a structurally related lipid, or a compound or molecule that affects the concentration or activity of anandamide or a structurally related lipid, in an amount sufficient for one use (i.e., one administration or one in vitro experiment), or it can comprise anandamide or a structurally related lipid, or a compound or molecule that affects the concentration or activity of anandamide or a structurally related lipid, in an amount sufficient for multiple uses (i.e., multiple administrations or multiple experiments).

In preferred embodiments, the compositions comprise anandamide or a structurally related lipid as the primary biologically active agent. In embodiments, the composition comprises other biologically active compounds, including, but not limited to, drugs, such as anti-inflammatory drugs, pain relievers (including local anaesthetics), and antibiotics.

Accordingly, the invention provides kits containing a compound or molecule that affects the activity of at least one vanilloid receptor. In embodiments, the kits contain anandamide or a structurally related lipid. The anandamide or structurally related lipid can be present in the kit as the sole component of the kit, or it can be present as part of a composition, alone or in combination with other compounds, solutions, or devices necessary or desirable for use of the anandamide or structurally related lipid. Thus, the anandamide or structurally related lipid can be present in the kit as the sole biologically active component or agent, or can be one of at least two biologically active components or agents. In embodiments, the kits contain a compound or molecule that affects the concentration or activity of anandamide or a structurally related lipid. The compound or molecule can be present in the kit as the sole component of the kit, or it can be present as part of a composition, alone or in combination with other compounds, solutions, or devices necessary or desirable for use of the compound or molecule. Thus, the compound or molecule can be present in the kit as the sole biologically active component or agent, or it can be one of at least two biologically active components or agents.

Thus, the kits of the invention can contain all the necessary compounds, solutions, and equipment for administration to an individual of anandamide, a structurally related lipid, or a compound or molecule that affects the in vivo activity or concentration of anandamide or a structurally related lipid. In addition, the kits of the invention can contain all the necessary compounds, solutions, and equipment for in vitro use of anandamide, a structurally related lipid, or a compound or molecule that affects the activity or concentration of anandamide or a structurally related lipid.

### EXAMPLES

The invention will now be described in more detail with reference to specific examples of the invention, which are not intended to be, and should not be construed as, limiting the scope of the invention in any way.

Methods:

Recording of tension. Experiments were performed on hepatic (400 μm outer diameter) and mesenteric (200 μm outer diameter) arteries from female Wistar-Hannover rats (200-250 g) and on basilar arteries (300 μm outer diameter) from male guinea pigs. Briefly, the arteries were cut into ring segments and mounted in tissue baths containing aerated physiological salt solution (5% CO₂ and 95% O₂; 37° C.; pH 7.4) as previously described (refs. 38, 43). All experiments were performed in the presence of N^{G}-nitro-L-arginine (0.3 mM) and indomethacin (10 μM) to eliminate any contribution of nitric oxide and cyclooxygenase products, respectively (refs. 38, 23). Relaxant responses were studied in preparations contracted with phenylephrine (rat hepatic and mesenteric arteries) and prostaglandin F_{2α} (guinea pig basilar artery) (refs. 38, 43). When stable contractions were obtained, agonists were added cumulatively to determine concentration-response relationships.

The incubation time with 8-37 CGRP, capsazepine and N-(piperidin-1-yl)-5-(4-chlorophenyl)-1-(2,4-dichlorophenyl)-4-methyl-1H-pyrazole-3-carboxamide hydrochloride (SR141716A) was 30 minutes. Some preparations were pre-treated with capsaicin for 1 hour followed by washout for 20 minutes. Each vessel segment was exposed to only one treatment. The experiments were carried out with the approval of the local ethics committee.

Measurement of CGRP and cyclic AMP. Segments of rat hepatic arteries were equilibrated for 1 hour in aerated physiological salt solution (5% CO₂ and 95% O₂; 37° C.; pH 7.4) containing N^{G}-nitro-L-arginine (0.3 mM) and indomethacin (10 μM).

CGRP: Preparations were transferred to Eppendorff tubes containing physiological salt solution with the addition of 0.05% BSA and the test drugs. After 10 minutes the segments were removed and the physiological salt solution was evaporated. The pellet was dissolved in RIA buffer and the amount of CGRP was determined by using rat ¹²⁵I-CGRP radioimmunoassay RIA kit (Peninsula labs).

Cyclic AMP: The amount of cyclic AMP after 5 min exposure to the test drugs was determined as previously described (ref. 44).

Immunohistochemistry. Rat hepatic arteries were fixed in formaldehyde for 4 hours, thereafter rinsed for three days and subsequently frozen (ref. 45). Cryostat sections were cut at a thickness of 10 μm and prepared for reaction with antibodies to CGRP (EURO-DIAGNOSTICA, Malmö, Sweden) at 1:320 dilution for 24 hours (room temperature; approximately 21-25° C.). CGRP-like immunoreactivity was visualized after exposure to FITC-conjugated goat anti-guinea pig IgG (Sigma, USA) at 1:80 dilution for 90 minutes (ref. 45).

Calculations and statistics. Relaxations are expressed as percentage reversal of the prevailing contraction (100% indicates a complete reversal to basal tension). Drug concentrations which elicited 50% relaxation (EC₅₀) and maximal relaxation (Eₘₐₓ) achieved were calculated as previously described (refs. 38, 43). Data are presented as means±s.e.mean (vertical lines in figures), and n indicates the number of vascular segments (animals) examined. pA₂ and slope values of the Shild plots were estimated according to the Jackknife method (ref. 46). Statistical analysis was performed by using Student's t-test (two-tailed), Mann-Whitney U-test, or analysis of variance (ANOVA) on log transformed values (cyclic AMP) followed by Bonferroni Dunn's post hoc test (Statview 4.12). Statistical significance was accepted when P<0.05.

Drugs. Anandamide, AM404, R(+)-methanandamide, WIN 55,212-2 (RBI); 1-arachidonylglycerol, 2-arachidonylglycerol (Cayman); HU 210, CP 55,940 (Tocris); palmitylethanolamide (Biomol); capsaicin, capsazepine (Fluka, Tocris); SR141716A (Sanofi Winthrop); and arachidonic acid (Sigma) were all dissolved in ethanol. Distilled water was used as solvent for acetylcholine chloride, L-phenylephrine hydrochloride, N^{G}-nitro-L-arginine, human 8-37 CGRP, ethanolamine hydrochloride (Sigma); human-α-CGRP (Peninsula); α-latrotoxin (Alomone), prostaglandin F_{2α} (Upjohn); and indomethacin (Confortid®, Dumex).

### Example 1

Anandamide is structurally related to capsaicin, the pungent ingredient in hot chili peppers (ref. 23), and to several of its congeners, including olvanil (N-vanillyloleamide) (ref. 24). These compounds all have an amide bond and an aliphatic side chain of varying length, but differ in their relative pungencies and analgetic properties (ref. 25) Capsaicin activates a subpopulation of primary sensory neurons which can then become refractory to subsequent stimuli (desensitization) and, depending on the concentration and length of exposure, can eventually degenerate (ref. 23). Such nerves are involved in non-adrenergic, non-cholinergic relaxation of smooth muscle as shown in, e.g., rat mesenteric arteries (ref. 26). Since capsaicin and related compounds induce vasodilatation (refs. 27, 28), we hypothesized that the vascular effects of anandamide and capsaicin may be mediated by a common mechanism, involving excitation of primary sensory nerve endings in the vessel wall with consequent release of vasodilator neuropeptides, such as CGRP.

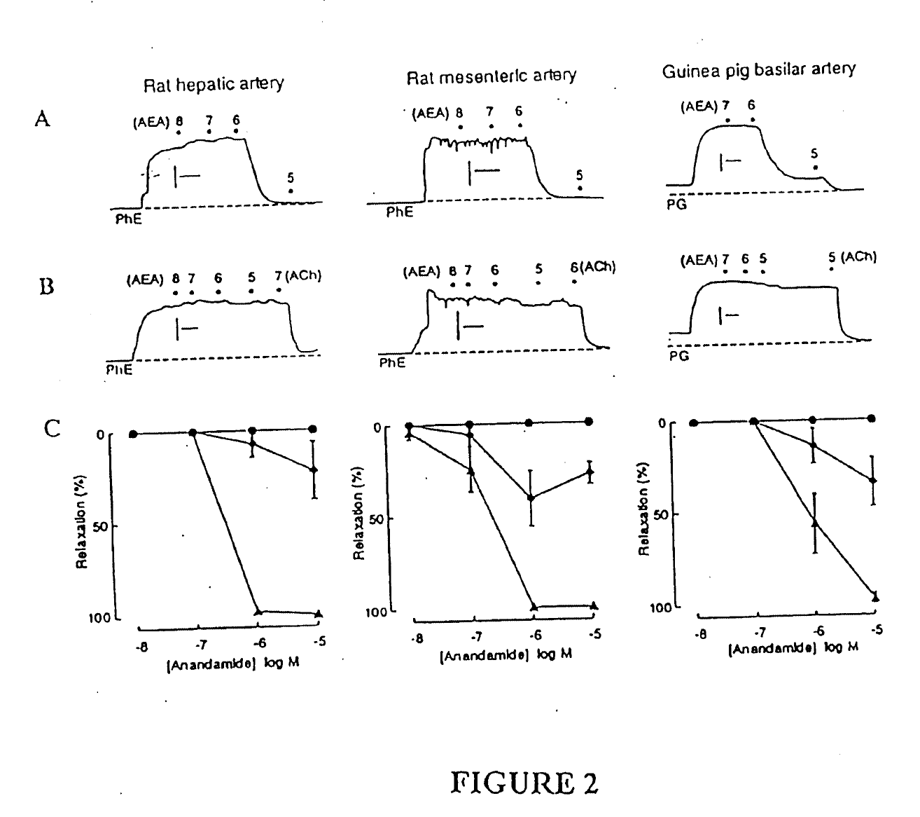

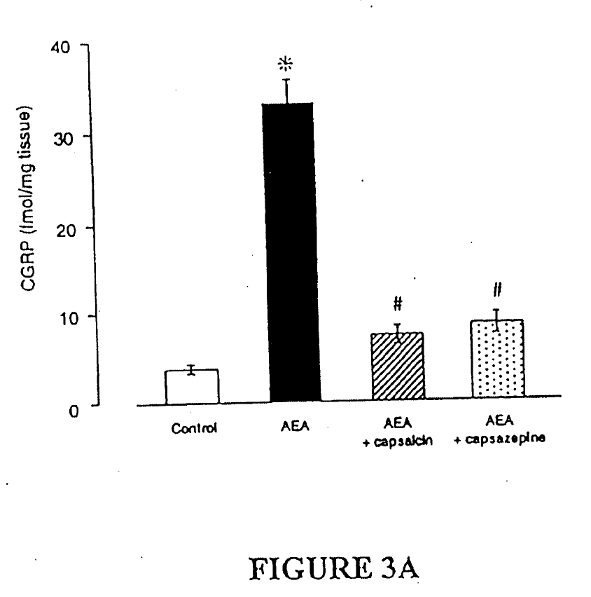

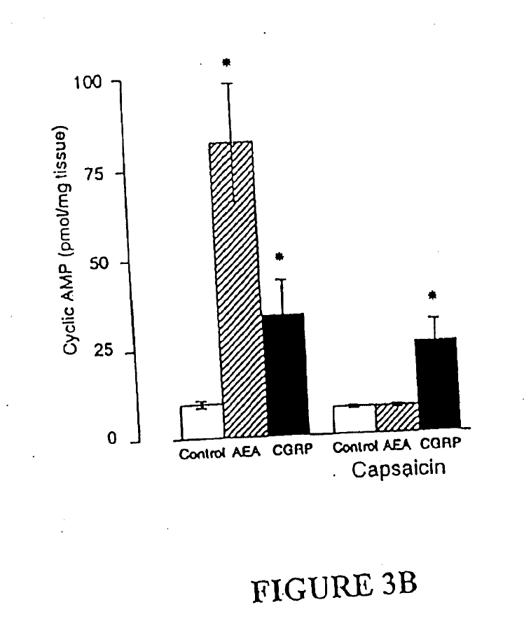

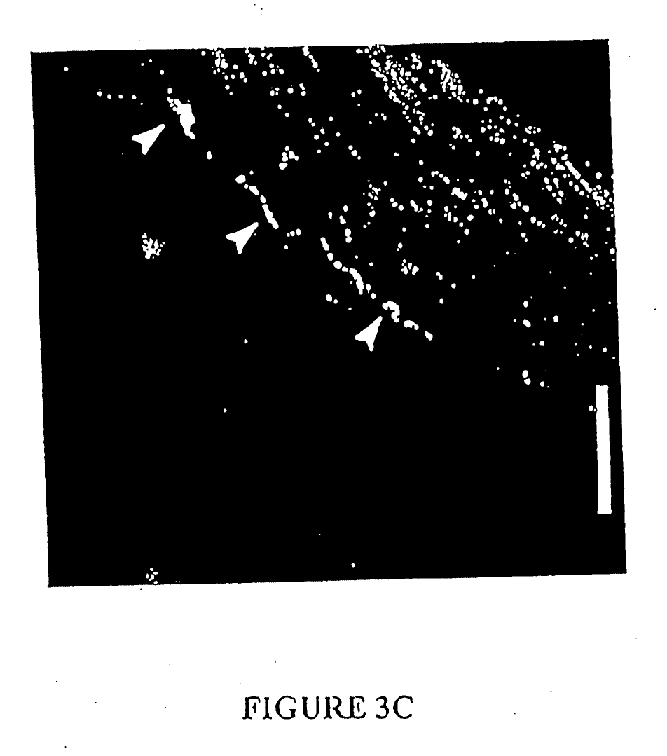

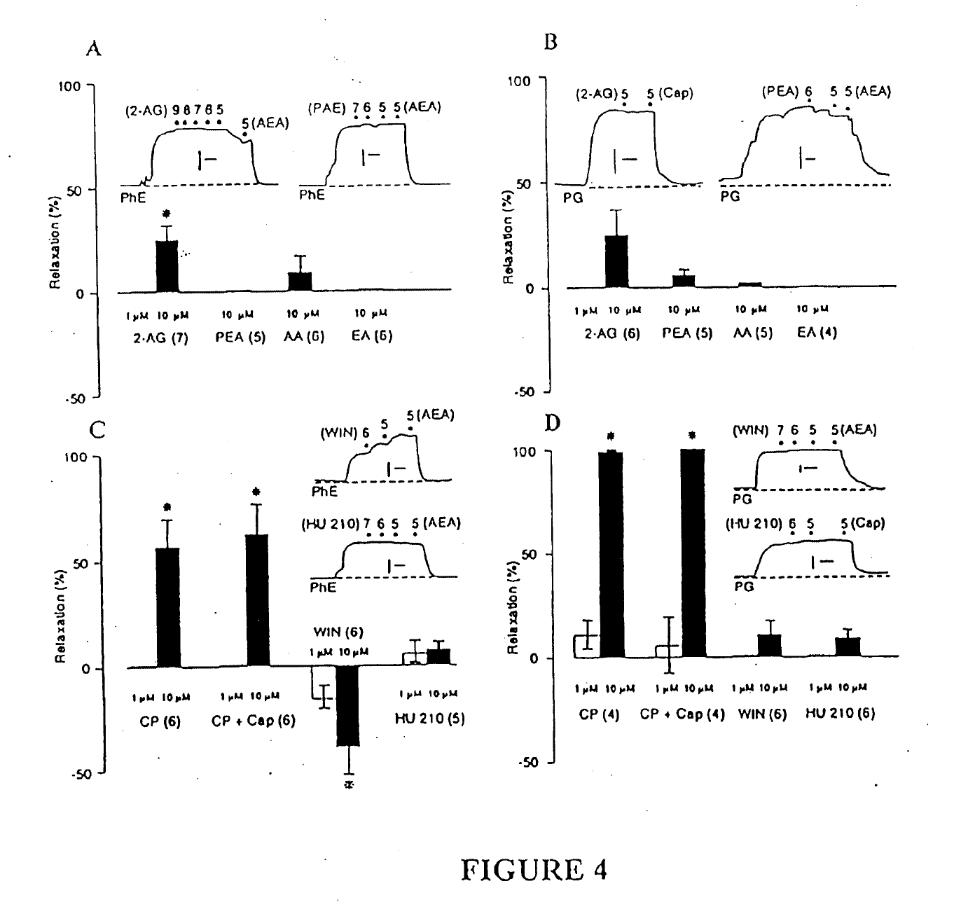

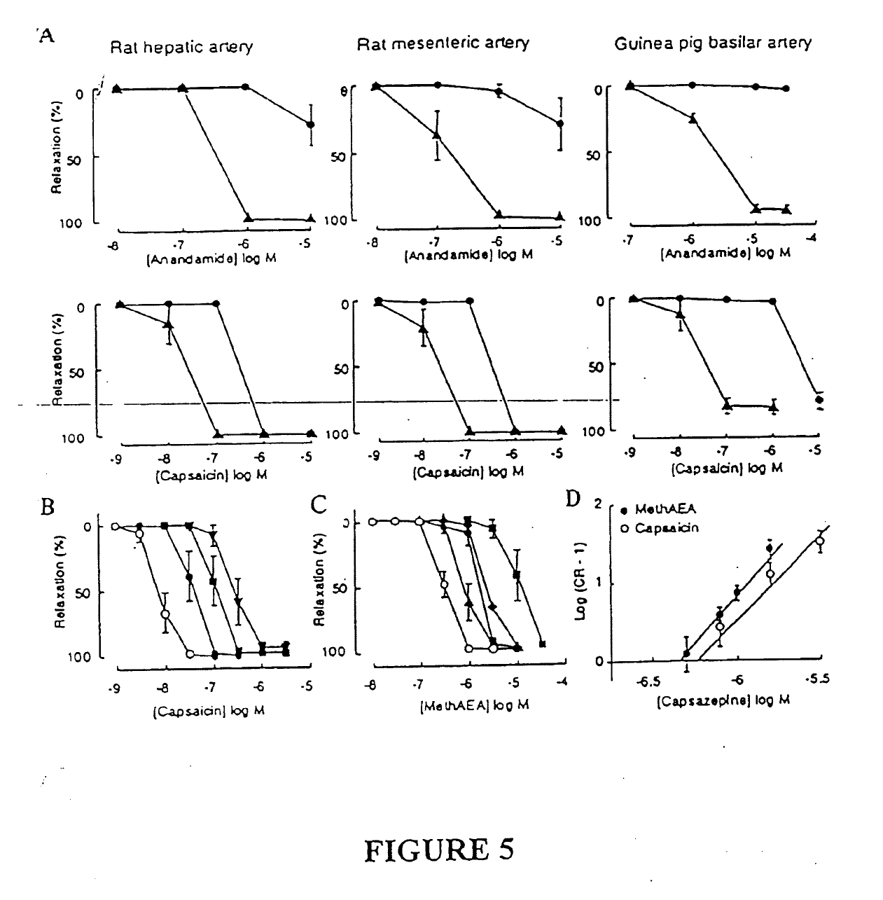

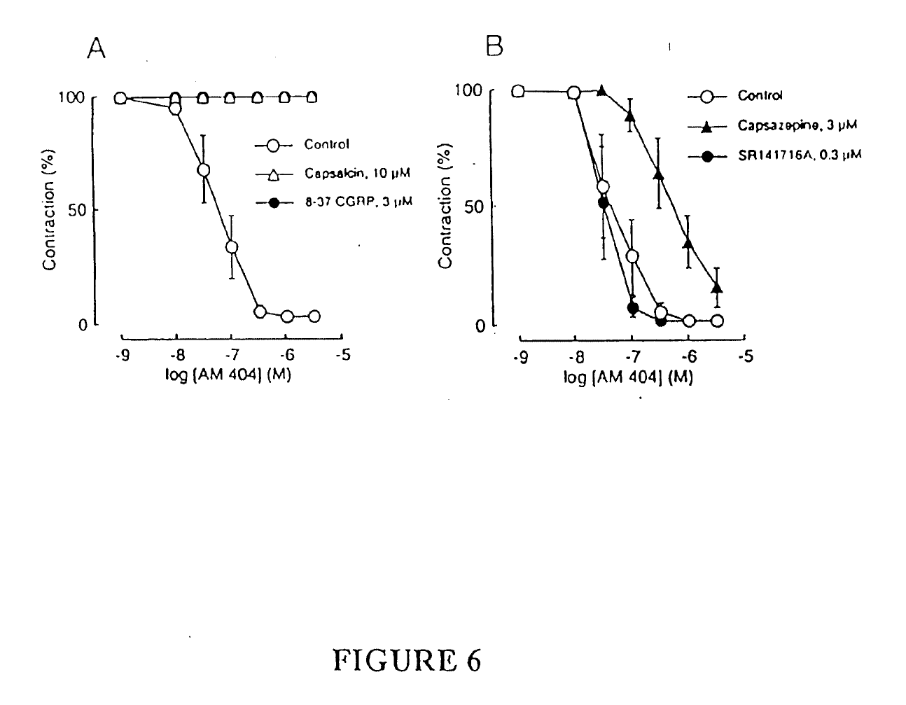

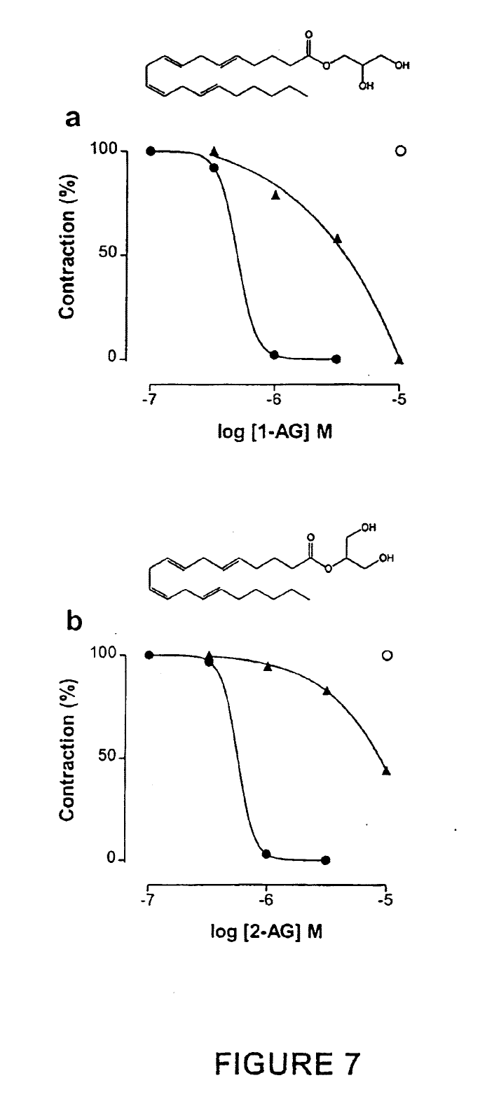

To test this hypothesis the effects of capsaicin and the selective CGRP receptor antagonist 8-37 CGRP (ref. 3) on anandamide-induced relaxation in rat hepatic and small mesenteric arteries and guinea pig basilar artery were examined. Pre-treatment with capsaicin to cause desensitization and/or neurotransmitter depletion of perivascular sensory nerves abolished the anandamide-induced relaxation in all three arteries (FIGS. 2A, 2B). The response to anandamide was also inhibited by the selective CGRP receptor antagonist 8-37 CGRP (FIG. 2C). As further shown in the rat hepatic artery, the relaxation induced by exogenous CGRP was unaffected by pre-treatment with capsaicin (10 μM), whereas 8-37 CGRP (2 μM) caused a significant rightward parallel shift of the concentration-relaxation curve for CGRP (control: pEC₅₀=9.56±0.04, Eₘₐₓ=100±1%; capsaicin: pEC₅₀=9.51±0.02, Eₘₐₓ=100±1%; 8-37 CGRP: pEC₅₀=8.54±0.13, Eₘₐₓ=100±1%; n=6).

In a previous study, a low concentration of anandamide was found to inhibit capsaicin-induced release of CGRP in rat spinal cord (ref. 29). Here it is shown that, at a concentration inducing vasodilation anandamide elicited a significant release of CGRP and an increase in tissue content of cyclic AMP in the rat hepatic artery (FIG. 3A, 3B), the latter effect presumably reflecting activation of CGRP receptors on the vasculature. Both these effects were absent in arterial segments pre-treated with capsaicin (FIGS. 3A, 3B). The cyclic AMP content was also increased by CGRP, but this effect was resistant to capsaicin (FIG. 3B). CGRP-like immunoreactive nerve fibers were visualized in the rat hepatic artery (FIG. 3C). Such nerves are also present in rat mesenteric (ref. 26) and guinea pig basilar (ref. 30) arteries. Collectively, these findings support our hypothesis that the vasodilator response to anandamide is mediated by release of CGRP from perivascular sensory nerves. The pronounced vasodilator effect of anandamide suggests that activation of these nerves is a powerful effector system for regulation of vascular tone.

Thus, the vasodilator response to anandamide in isolated arteries is capsaicin-sensitive and accompanied by a release of calcitonin gene-related peptide (CGRP) and an increase in cyclic AMP. The selective CGRP receptor antagonist 8-37 CGRP (ref.

3), but not the CB1 receptor blocker SR141716A (ref. 4), inhibited the vasodilator effect of anandamide. Other endogenous (2-arachidonylglycerol, palmitylethanolamide) and synthetic (HU 210, WIN 55,212-2, CP 55,940) CB1 and CB2 receptor agonists (ref. 5) were unable to mimic the action of anandamide. In addition, the selective vanilloid receptor antagonist capsazepine (refs. 6, 7) inhibited the anandamide-induced vasodilator response and outflow of CGRP. The results presented here indicate that activation of vanilloid receptors on perivascular sensory nerves and subsequent release of the vasodilator peptide CGRP causes the vasodilator effect of anandamide. This if fully consistent with the vanilloid receptor being a molecular target for endogenous anandamide in the nervous and cardiovascular systems.

### Example 2

To elucidate the role of CB receptors in perivascular sensory nerve activation, the vascular effects of anandamide were compared with those of a series of endogenous (palmitylethanolamide, 2-arachidonylglycerol) and synthetic (HU 210, WIN 55,212-2, CP 55,940) CB receptor agonists with varying selectivity for CB1 and CB2 receptors (refs. 5, 15, 16, 31). HU 210, WIN 55,212-2 and CP 55,940 are at least one order of magnitude more potent than anandamide as agonists of CB1 and CB2 receptors (ref. 16). In rat hepatic and guinea pig basilar arteries, palmitylethanolamide, HU 210 and WIN 55,212-2 did not produce any relaxation, whereas 2-arachidonylglycerol induced a small (rat hepatic artery) or inconsistent (guinea pig basilar artery) relaxation at 10 μM (FIG. 4). CP 55,940 elicited a significant relaxation in both arteries, but this effect was resistant to pre-treatment with capsaicin and occurred only at a concentration of 10 μM (FIGS. 3C, D). The failure of these different CB receptor agonists to mimic the action of anandamide at concentrations which should fully activate CB1 and/or CB2 receptors suggests that these receptors are not involved in the vasodilator effect of anandamide.

The competitive CB1 receptor antagonist SR141716A (0.3 μM), which in nanomolar concentrations binds to CB1 receptors in brain (Kᵢ=2 nM) (ref. 4) and inhibits the effects of CB1 receptor agonists in the periphery (ref. 32), did not attenuate the vasodilator responses to anandamide in rat hepatic (control: pEC₅₀=6.45±0.11, Eₘₐₓ=100±1%; SR141716A: pEC₅₀=6.44±0.12, Eₘₐₓ97±1%; n=5) and guinea pig basilar (control: pEC₅₀=6.02±0.11, Eₘₐₓ=92±3%; SR141716A: pFC₅₀=6.11±0.10, Eₘₐₓ=91±2%; n=5-8) arteries, and methanandamide in the rat hepatic artery (control: pEC₅₀=6.50±0.08, Eₘₐₓ99±1%; SR141716A: pEC₅₀=6.30±0.04, Eₘₐₓ=99±1%; n=5). However, high concentrations of SR141716A (≧3 μM) were able to inhibit the anandamide-induced relaxation in rat hepatic (ref. 20) and mesenteric (ref. 22), and guinea pig basilar (ref. 33) arteries, but the mechanism of this action is unclear. Such high concentrations of SR141716A seem to have effects unrelated to inhibition of CB1 and CB2 receptors (refs. 32, 34, 35). This is supported by the present findings, showing that the vasodilator response to capsaicin, which does not bind to CB1 receptors (ref. 36), is significantly inhibited by SR141716A (10 μM) in the rat hepatic artery (control: pEC₅=8.37±0.13, Eₘₐₓ=99±1%; SR141716A: pEC₅₀=7.42±0.12, Eₘₐₓ=90±3%; n=4).

It has been suggested that enzymatic cleavage of anandamide by a specific amidohydrolase (ref. 31) to arachidonic acid and ethanolamine, and subsequent formation of vasodilator eicosanoids, such as prostacyclin and eicosatrienoic acids, is responsible for the anandamide-induced relaxation in bovine coronary artery (ref. 34). However, this relaxation was endothelium-dependent in contrast to the vasodilator response to anandamide in rat hepatic (ref 20) and mesenteric (ref. 22), and guinea pig basilar arteries. Cyclooxygenase products can be ruled out by the present data, since indomethacin was present in all experiments, and eicosatrienoic acids are unable to relax rat hepatic (ref. 37) and guinea pig basilar (ref. 38) arteries. Furthermore, arachidonic acid (10 μM) and ethanolamine (10 μM) proved to be inactive in these arteries (FIGS. 4A, 4B), whereas methanandamide, which is resistant to enzymatic hydrolysis (ref. 39), was at least equipotent with anandamide as a vasodilator in rat hepatic (methanandamide: pEC₅₀=6.53±0.08, Eₘₐₓ=97±1%; n=8; anandamide: pEC₅₀=6.49±0.01, Eₘₐₓ=100±1%; n=6) and mesenteric (ref. 35) arteries. These findings clearly indicate that anandamide itself is responsible for the vasodilator activity.

### Example 3

Capsaicin and other vanilloid compounds exert their actions by binding to specific vanilloid receptors on primary sensory neurons. Molecular studies have shown that the cloned vanilloid receptor (VR1) is a calcium-permeable, non-selective cation channel that is gated by noxious heat or extracellular protons (ref. 8). As recently shown, olvanil (but not capsaicin) binds to both the anandamide transporter and the CB1 receptor, indicating that capsaicin-like compounds and endocannabinoids can indeed be recognized by the same proteins (ref. 36). VR1 is most closely related to members of the transient receptor potential (TRP) channel family, and recent studies have shown that membrane-derived second messengers, such as diacylglycerol or arachidonic acid, can activate certain TRP channel subtypes at micromolar concentrations (refs. 40, 41).

To determine whether native vanilloid receptors might be involved in the action of anandamide, the effect of capsazepine, a competitive, vanilloid receptor antagonist (refs. 6, 7), on the vasodilator responses to anandamide/methanandamide and capsaicin was examined. As shown in rat hepatic and mesenteric, and guinea pig basilar arteries, capsazepine significantly inhibited the vasodilator effects of capsaicin or anandamide (FIG. 5A). Further experiments on the rat hepatic artery showed that capsazepine produced a rightward parallel shift the of the concentration-response curves for both capsaicin and methanandamide without affecting maximal response, consistent with a competitive mode of inhibition (FIGS. 5B, 5C). The Schild plots for capsazepine using capsaicin and methanandamide as agonists were not significantly different, revealing pA₂ values of 6.24±0.14 and 6.34±0.06, respectively (FIG. 5D), strongly favoring an identical site of action of capsaicin and of methanandamide. Capsazepine also inhibited the anandamide-induced release of CGRP from capsaicin-sensitive nerves (FIG. 3A). The vasodilator response to α-latrotoxin (1 nM), a neurotoxin that causes neurotransmitter release after binding to presynaptic nerve terminals (ref. 42), was unaffected by capsazepine (3 μM) in the rat hepatic artery, but was abolished by CGRP 8-37 (2 μM) or capsaicin pre-treatment (control: 98±1%, n=12; capsazepine: 93±2%, n=6; CGRP 8-37: −8±3%, n=6; capsaicin: −16±8%, n=7), indicating that capsazepine does not act via depletion of neurotransmitter from sensory nerves. Furthermore, capsazepine (3 μM) had no effect on the CGRP-induced relaxation in rat hepatic (control: pEC₅₀=9.46±0.01, Eₘₐₓ=100±1%; capsazepine: pEC₅₀=9.39±0.04, Eₘₐₓ=100±1%, n=6), mesenteric (control: pEC₅₀=10.4±0.04, Eₘₐₓ99±1%; capsazepine: pEC₅₀ 10.3±0.12, Eₘₐₓ=99±1%, n=4) and guinea pig basilar (control: pEC₅₀=8.72±0.16, Eₘₐₓ=98±1%; capsazepine: pEC₅₀=8.70±0.15, Eₘₐₓ=99±1%, n=4-5) arteries, confirming the specificity of capsazepine.

### Example 4

The anandamide transport inhibitor AM404 has been developed to prevent inactivation of anandamide by a cellular re-uptake mechanism and thereby prolong the biological effects of anandamide (ref. 52). This mechanism of inactivation of anandamide has been suggested to be of biological importance, e.g., in blood pressure regulation, since AM404 potentiated hypotensive responses to anandamide in guinea-pigs (ref 53).

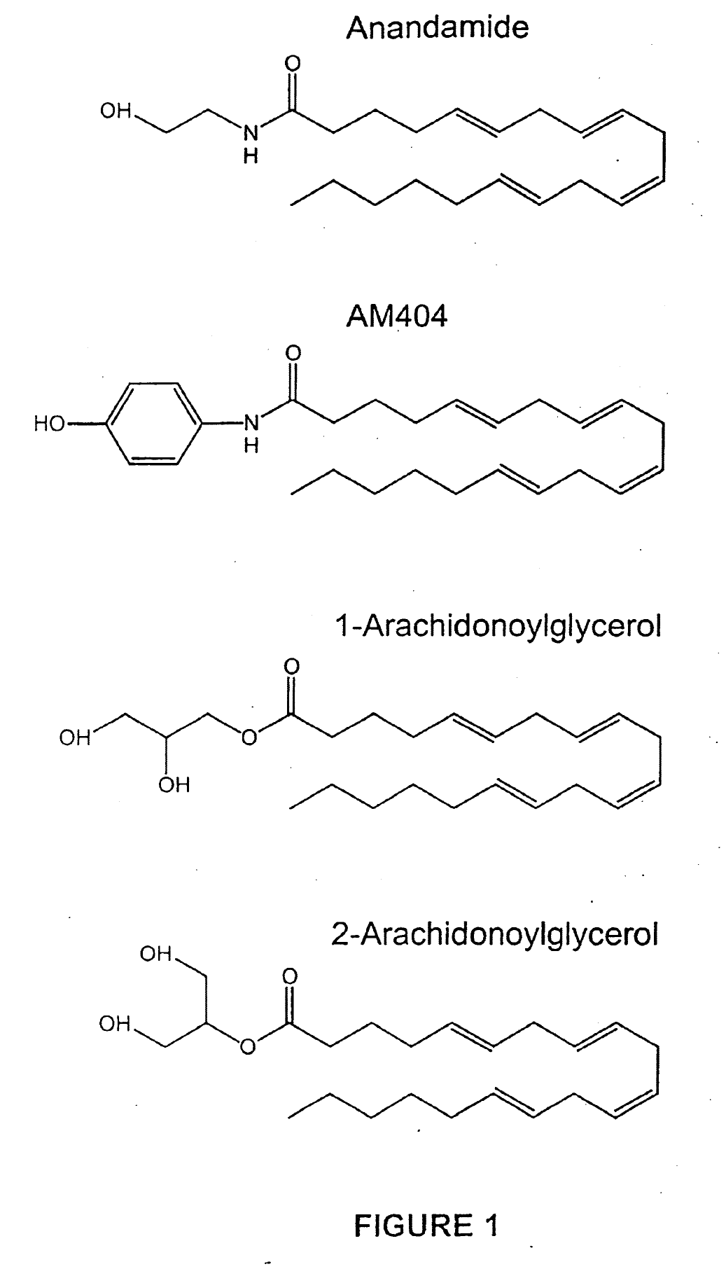

Initially, we attempted to investigate the influence of the anandamide transporter on the vasodilator action of anandamide in rat isolated hepatic arteries. Anandamide relaxes this blood vessel via activation of vanilloid receptors present on perivascular sensory nerves and the subsequent release of vasodilator peptides such as CGRP (ref. 54). However, we found that AM404 did not potentiate the vasodilation induced by anandamide, but rather inhibited this response. The unsaturated fatty acyl chain combined with a vanillyl-like moiety makes AM404 structurally similar to both anandamide (FIG. 1) and capsaicin. As reported below, we therefore explored the possibility that this compound acts as a vanilloid receptor agonist, causing vasodilation via activation of capsaicin-sensitive sensory nerves.

Effect of Capsaicin and 8-37 CGRP on AM404-Induced Vasodilation

AM404 induced concentration-dependent relaxations in hepatic arteries of the rat (FIGS. 6A, 6B). The pEC₅₀ and Eₘₐₓ values were 7.4±0.1 and 97±2%, respectively (n=10). Pre-treatment of preparations with capsaicin (10 μM) abolished AM404 induced relaxations (FIG. 6A). Likewise, the CGRP receptor antagonist 8-37 CGRP (3 μM) abolished relaxations induced by AM404 (FIG. 6A).

Effect of Capsazepine and SR141716A on AM404-Induced Vasodilation

The vanilloid receptor antagonist capsazepine (3 μM) caused a significant right-ward shift of the concentration-response curve for AM404 (FIG. 6B). The pEC₅₀ values were 7.3±0.2 and 6.4±0.2 in the absence and in the presence of capsazepine, respectively (n=5-7). In contrast, the cannabinoid CBI receptor antagonist SR141716A (0.3 μM) was without effect on AM404-induced relaxations (FIG. 6B).

The present example shows that AM404 is a vasodilator and activator of vanilloid receptors. Pre-treatment with capsaicin abolished AM404-induced relaxations, indicating that sensory nerves are involved in the vasodilator action of AM404. The fact that the CGRP receptor antagonist 8-37 CGRP abolished vasodilation induced by AM404 suggests that CGRP is the mediator of such relaxations. Indeed, anandamide, which releases CGRP from sensory nerves in the rat hepatic artery, is unable to cause vasodilation when the CGRP receptor is blocked by 8-37 CGRP (ref. 54). In the present study, the relaxant effect of AM404 was inhibited by the vanilloid receptor antagonist capsazepine (ref. 6), which competitively inhibits relaxations evoked by capsaicin and methanandamide in the rat hepatic artery (ref. 54). The right-ward shift of the concentration-response curve to AM404 caused by 3 μM capsazepine is of the same magnitude a when capsaicin is used as an agonist (ref. 54), indicating that AM404 is acting on vanilloid receptors. Capsazepine also blocks currents through VR1 induced by capsaicin and anandamide (refs. 8, 54).

It is possible that AM404 indirectly causes vasodilation by inhibiting the anandamide transporter, leading to increased levels of endogenous anandamide and subsequent vanilloid receptor activation. However, the vasodilator action of AM404 in the rat hepatic artery occurs at lower concentrations than those shown to inhibit the anandamide transporter (refs. 52, 56, 36). Thus, an action of AM404 on cannabinoid CB1 receptors seems unlikely since SR141716A, a selective cannabinoid CB1 receptor antagonist when used at submicromolar concentrations (see ref. 5) was without effect on relaxations elicited by AM404. This is in agreement with findings showing that AM404, in contrast to cannabinoid CB1 receptor agonists, does not inhibit forskolin-induced cyclic AMP accumulation (ref. 52). Thus, the phenolic moiety of AM404 apparently decreases the affinity for cannabinoid CB1 receptors-Kᵢ values being 78 nM for anandamide and 1760 nM for AM404 (ref. 57). The affinity of AM404 to the cannabinoid CB2 receptor is even less (ref. 57). Interestingly, the phenolic moiety may improve the interaction with the vanilloid receptor, since AM404 is almost 10 times more potent as a vasodilator compared to anandamide in the rat hepatic artery (ref. 54).

Thus, the present example shows that AM404 is similar in activity to capsaicin and anandamide and can be regarded as a vanilloid receptor ligand. It is important to note that this feature of AM404 might complicate its use as a pharmacological tool to evaluate the role of the anandamide transporter in test models containing vanilloid receptors.

### Example 5

The present example shows that 1-arachidonylglycerol and 2-arachidonylglycerol are vasodilators via activation of vanilloid receptors on perivascular sensory nerves in rat mesenteric arteries (FIG. 7). Pre-treatment with capsaicin abolished, and the vanilloid receptor antagonist capsazepine (refs. 6, 54) inhibited, the relaxant effects of 1-arachidonylglycerol and 2-arachidonylglycerol.

It is intended that the specification and examples be considered as exemplary only, with a true scope and spirit of the invention being indicated by the following claims. All references cited herein are hereby incorporated in their entireties.

### REFERENCES

1. Devane, W. A. et al., Isolation and structure of a brain constituent that binds to the cannabinoid receptor., Science 258, 1946-1949 (1992).

2. Randall, M. D. & Kendall, D. A., Endocannabinoids: a new class of vasoactive substances, TIPS 19, 55-58 (1998).

3. Han, S.-P., Naes, L. & Westfall, T. C., Inhibition of periarterial nerve stimulation-induced vasodilation of the mesenteric arterial bed by CGRP (8-37) and CGRP receptor desensitization, Biochem Biophys Res Com 168, 786-791 (1990).

4. Rinaldi-Carmona, M. et al., SR141716A, a potent and secretive antagonist of the brain cannabinoid receptors., FEBS Letters 350, 240-244 (1994).

5. Pertwee, R. G., Pharmacology of cannabinoid CB₁ and CB₂ receptors., Pharmacol Ther 74, 129-180 (1997).

6. Bevan, S. et al., Capsazepine: a competitive antagonist of the sensory neurone excitant capsaicin., British Journal of Pharmacology 107, 544-552 (1992).

7. Wardle, K. A., Ranson, J. & Sanger, G. J., Pharmacological characterization of the vallinoid receptor in the rat dorsal spinal chord., British Journal of Pharmacology 121, 1012-1016 (1997).

8. Caterina, M. J. et al., The capsaicin receptor: a heat-activated ion channel in the pain pathway., Nature 389, 816-824 (1997).

9. Deutsch, D. G. & Chin, S. A., Enzymatic synthesis and degradation of anandamide, a cannabinoid receptor agonist., Biochem. Pharmacol. 46, 791-796 (1993).

10. Di Marzo, V. et al., Formation and inactivation of endogenous cannabinoid anandamide in central neurons., Nature 372, 686-691 (1994).

11. Deutsch, D. G. et al., Production and physiological actions of anandamide in the vasculature of the rat kidney., Journal of Clinical Investigation 100, 1538-1546 (1997).

12. Di Marzo, V., De Petrocellis, L., Sepe, N. & Buono, A., Biosynthesis of anandamide and related acylethanolamides in mouse J774 macrophages and N₁₈ neuroblastoma cells., Biochemical Journal 316, 977-984 (1996).

13. Wagner, J. A. et al., Activation of peripheral CB, cannabinoid receptors in haemorrhagic shock., Nature 390, 518-521 (1997).

14. Varga, K., Wagner, J. A., Bridgen, D. T. & Kunos, G., Platelet- and macrophage-derived endogenous cannabinoids are involved in endotoxin-induced hypotension., FASEB Journal 12, 1035-1044 (1998).

15. Hillard, C. J. & Campbell, W. B., Biochemistry and pharmacology of arachidonylethanolamide, a putative endogenous cannabinoid., Journal of Lipid Research 38, 2383-2398 (1997).

16. Felder, C. C. et al., Comparison of the pharmacology and signal transduction of the human cannabinoid CB₁ and CB₂ receptors., Molecular Pharmacology 48, 443-450 (1995).

17. Sugiura, T. et al., Detection of an endogenous cannabimimetic molecule 2-arachidonoylglycerol, and cannabinoid CB1 receptor mRNA in human vascular cells: is 2-arachidonoylglycerol a possible vasomodulator?, Biochemical and Biophysical Research Communications 243, 838-843 (1998).

18. Varga, K., Lake, K. D., Huangfu, D., Guyenet, P. G. & Kunos, G., Mechanism of the hypotensive action af anandamide in anesthetized rats. Hypertension 28, 682-868 (1996).

19. Plane, F., Holland, M., Waldron, G. J., Garland, C. J. & Boyle, J. P., Evidence that anandamide and EDHF act via different mechanisms in rat isolated mesenteric arteries., British Journal of Pharmacology 121, 1509-1511 (1997).

20. Zygmunt, P. M. et al., Studies on the effects of anandamide in rat hepatic artery., British Journal of Pharmacology 122, 1679-1686 (1997).

21. Chataigneau, T. et al., Cannabinoid CB1 receptor and endothelium-dependent hyperpolarization in guinea-pig carotid, rat mesenteric and porcine coronary arteries., British Journal of Pharmacology 123, 968-974 (1998).

22. White, R. & Hiley, C. R., The actions of some cannabinoid receptor ligands in the rat isolated mesenteric artery., British Journal of Pharmacology 125, 533-541 (1998).

23. Szallasi, A., The vanilloid (capsaicin) receptor: receptor types and species differences., General Pharmacology 25, 223-243 (1994).

24. Walpole, C. S. J. et al., Analogues of capsaicin with agonist activity as novel analgesic agents: structure-activity studies: part 3. The hydrophobic side-chain “C-region”., Journal of Medical Chemistry 36, 2381-2389 (1993).

25. Wrigglesworm, R. et al., Analogues of capsaicin with agonist activity as novel analgesic agents: Structure-activity studies. 4. Potent, orally active analgesics., J. Med. Chem. 39, 4942-4951 (1996).

26. Kawasaki, H., Takasaki, K., Saito, A. & Goto, K., Calcitonin gene-related peptide acts as a novel vasodilator neurotransmitter in mesenteric resistance vessels of the rat., Nature 335, 164-167 (1988).

27. Hughes, S. R., Buckley, T. L. & Brain, S. D., Olvanil: more potent than capsaicin at stimulating the efferent function of sensory nerves., European Journal of Pharmacology 219, 481-484 (1992).

28. Franco-Cereceda, A., Resiniferatoxin-, capsaicin- and CGRP-evoked porcine coronary vasodilatation is independent of EDRF mechanisms but antagonized by CGRP(8-37)., Acta Physiologica Scandinavica 143, 331-337 (1991).

29. Richardson, J. D., Aanonsen, L. & Hargreaves, K. M., Antihyperalgesic effects of spinal cannabinoids., European Journal of Pharmacology 345, 145-153 (1998).

30. Edvinsson, L., Ekman, R., Jansen, I., McCulloch, J. & Uddman, R., Calcitonin gene-related peptide and cerebral blood vessels: distribution and vasomotor effects., Journal of Cerebral Blood Flow and Metabolism 7, 720-728 (1987).

31. Di Marzo, V., Melck, D., Bisogno, T. & De Petrocellis, L., Endocannabinoids: endogenous cannabinoid receptor ligands with neuromodulatory action., Trends Neurosci. 21, 521-528 (1998).

32. Coutts, A. A. & Pertwee, R. G., Inhibition by cannabinoid receptor agonists of acetylcholine release from the guinea-pig myenteric plexus., Br. J. Pharmacol. 121, 1557-1566 (1997).

33. Petersson, J., Zygmunt, P. M., Jösson, P. & Högestätt, E. D., Involvement of derivatives of arachidonic acid in endothelium-dependent relaxations mediated by EDHF in the guinea-pig basilar artery., British Journal of Pharmacology Proceedings Supplement 122, 401P (1997).

34. Pratt, P. F., Hillard, C. J., Edgemond, W. S. & Campbell, W. B., N-arachidonylethanolamide relaxation of bovine coronary artery is not mediated by CB1 cannabinoid receptor., American Journal of Physiology 43, H375-H381 (1998).

35. White, R. & Hiley, C. R., The actions of the cannabinoid receptor antagonist, SR 141716A, in the rat isolated mesenteric artery., British Journal of Pharmacology 125, 689-696 (1998).

36. Di Marzo, V., et al., Interactions between synthetic vanilloids and the endogenous cannabinoid system., FEBS Letters 436, 449-454 (1998).

37. Zygmunt, P. M., Edwards, G., Weston, A. H., Davis, S. C. & Högestätt, E. D., Effects of cytochrome P450 inhibitors on EDHF-mediated relaxation in the rat hepatic artery., British Journal of Pharmacology 118, 1147-1152 (1996).

38. Petersson, J., Zygmunt, P. M., Jönsson, P. & Högestätt, E. D., Characterization of endothelium-dependent relaxation in guinea-pig basilar artery—effects of hypoxia and role of cytochrome P₄₅₀ mono-oxygenase., Journal of Vascular Research 35, 285-294 (1998).

39. Abadji, v. et al., (R)-methanandamide: A chiral novel anandamide possessing higher potency and metabolic stability., J. Med. Chem. 37, 1889-1893 (1994).

40. Chyb, S., Raghu, P. & Hardie, R., Polyunsaturated fatty acids activate the drosophila light-sensitive channels TRP and TRPL., Nature 397, 255-259 (1999).

41. Hofmann, T. et al., Direct activation of human TRPC6 and TRPC3 channels by diacylglycerol., Nature 397, 259-263 (1999).

42. Geppert, M. et al., Neurexin Ia is a major a-latrotoxin receptor that cooperates in a-latrotoxin action., J. Biol. Chem. 273, 1705-1710 (1998).

43. Zygmunt, P. M., Plane, F., Paulsson, M., Garland, C. J. & Högestätt, E. D., Interactions between endothelium-derived relaxing factors in the rat hepatic artery: focus on regulation of EDHF., British Journal of Pharmacology 124, 992-1000 (1998).

44. Zygmunt, P. M., Grundemar, L. & Högestätt, E. D., Endothelium-dependent relaxation resistant to Nw-nitro-L-arginine in the rat hepatic artery and aorta., Acta Physiologica Scandinavica 152, 107-114 (1994).

45. Persson, K., Alm, P., Johansson, K., Larsson, B. & Andersson, K.-E., Co-existence of nitrergic, peptidergic and acetylcholine esterase-positive nerves in the pig lower urinary tract., Journal of the Autonomic Nervous System 52, 225-236 (1995).

46. Gray, H. L. & Schucany, W. R., The Generalized Jacknife Statistic., Dekker, New York (1972).

47. Holzer P., Capsaicin: cellular targets, mechanisms of action, and selectivity for thin sensory neurons., Pharmacol Rev 43, 143-201 (1991).

48. Maggi C A., Capsaicin and primary afferent neurons: from basic science to human therapy., J Autonom Nerv Syst 33, 1-14 (1991).

49. Maggi C A, Meli A., The sensory-effecernt function of capsaicin-sensitive sensory neuron. Gen Pharmacal 19, 1-43 (1988)

50. Szolcsanyi J, Jancso-Gabor A., Sensory effects of capsaicin congeners I. Relationship between chemical structure and pain-producing potency of pungent agents., Arzneimittelforschung 25, 1877-1881 (1975).

51. Tominaga K, Caterina M J, Mahnberg A B, Rosen T A, Gilbert H, Skinner K, Raumann B E, Basbaum A I, Julius D., The cloned capsaicin receptor integrates multiple pain-producing stimuli., Neuron 21, 531-543 (1998).

52. Beltramo, M., Stella, N., Calignano, A., Lin, S. Y., Makriyannis, A., Piomelli, D., Functional role of high-affinity anandamide transport, as revealed by selective inhibition., Science 277, 1094-1097 (1997).

53. Calignano, A., La Rana, G., Beltramo, M., Makriyannis, A., Piomelli, D., Potentiation of anandamide hypotension by the transport inhibitor, AM404., Eur. J. Pharmacol. 337, R1-R2 (1997).

54. Zygmunt, P. M., Petersson, J., Andersson, D. A., Chuang, H-h., Sörg\dot{a}rd, M., Di Marzo, V., Julius, D., Högestätt, E. D., Vanilloid receptors on sensory nerves mediate the vasodilator action of anandamide., Nature 400, 452-457 (1999).

55. Melck, D., Bisogno, T., De Petrocellis, L., Chuang, H-h., Julius, D., Bifulco, M., Di Marzo, V., Unsaturated long-chain N-acyl-vanillyl-amides (N-AVAMs) vanilloid receptor ligands that inhibit anandamide-facilitated transport and bind to CB1 cannabinoid receptors., Biochem. Biophys. Res. Commim. 262, 275-284 (1999).

56. Beltramo, M., Piomelli, D., Anandamide transport inhibition by the vanilloid agonist olvanil., Eur. J. Pharmacol. 364, 75-78 (1999).

57. Khanolkar, A. D., Abadji, V., Lin, S., Hill, W. A. G., Taha, G., Abouzid, K., Meng, Z., Fan, P., Makriyannis, A., Head group analogs of arachidonylethanolamide, the endogenous cannabinoid ligand., J. Med. Chem. 39, 4515-4519 (1996).

58. Walpole et al., J. Med. Chem. 36:2373-2380 (1993).

59. B [symbol] ndgaard, H., ed., Design of Prodrugs, Elsevier Science Publ. B. V., New York, N. Y., (1985).

What is claimed is:

1. A method of treating an individual suffering from, or suspected of having a high risk of developing, at least one disease or disorder, or a symptom of at least one disease or disorder, associated with abnormal activity of at least one vanilloid receptor, wherein said method comprises administering a compound that is structurally related to anandamide, AM404, 1-arachidonylglycerol, or 2-arachidonylglycerol to the individual in an amount sufficient to result in modulation of the activity of at least one vanilloid receptor,
wherein the compound that is structurally related to anandamide, AM404, 1-arachidonylglycerol, or 2-arachidonylglycerol can be represented by formula (I) or formula (II),
wherein formula (I) is:
A-B-C
in which A can be represented by 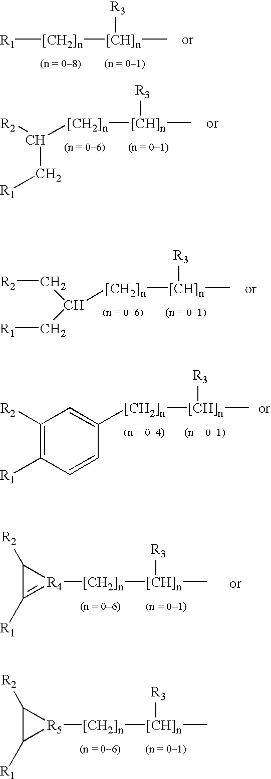
wherein R₁ can be any of the following substituents: —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br, and I; and
wherein R₂ can be any of the following substituents: —H, —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H5, —CH₂SCH₃, —C₂H₅SCH₃, —NO₂, —OCH₂NH₂, —OC₂H₅NH₂, Cl, F, Br and I;
wherein R₂ is not hydrogen when R₁ is alkoxy; and wherein any hydroxy group of R₁ and R₂ may be protected by a metabolically deprotectable protecting group to provide —OH in situ; and
wherein R₃ can be —H, —CH₃, —C₂H₅, and CF₃;
wherein R₄ can be 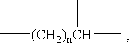
wherein n is 0-4; and
wherein R₅ can be 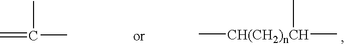
wherein n is 0-3;
in which B can be represented by —NHC(O)—, —NHC(S)—, —NHC(O)NH—, —NHS(O)—, —C(O)O—, —C(O)S—, —C(S)O—, —NHS—, —C(O)NH—, —C(S)NH—, —NHC(S)NH—, —S(O)NH—, —OC(O)—, —SC(O)—, —OC(S)— or —SNH—; and
in which C can be represented by an unsaturated straight or branched hydrocarbon chain, containing 6 to 24 carbon atoms, preferably 12 to 22 carbon atoms, and at least one double bond; and
wherein formula (II) is:
D-E-C
in which D can be represented by 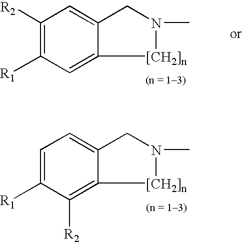
wherein R₁ can be any of the following substituents: —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br, and I; and
wherein R₂ can be any of the following substituents: —H, —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br and I;
wherein R₂ is not hydrogen when R₁ is alkoxy; and wherein any hydroxy group of R₁ and R₂ may be protected by a metabolically deprotectable protecting group to provide —OH in situ; and
in which E can be represented by —C(O)—, —C(S)—, —C(O)NH—, —C(S)NH—, —S(O)—, —S—, —O—, —C(O)O—, —C(O)S—, —OC(O)—, or C(S)O—; and
in which C can be represented by an unsaturated straight or branched hydrocarbon chain, containing 6 to 24 carbon atoms, preferably 12 to 22 carbon atoms, and at least one double bond.

2. The method of claim 1, wherein said at least one vanilloid receptor is vanilloid receptor 1 (VR1).

3. The method of claim 1, wherein said method treats a disease, disorder, or symptom selected from the group consisting of inflammation, pain, allergy or autoimmune disease, organ dysfunction, infection, and wounds.

4. The method of claim 3, wherein said inflammation is selected from the group consisting of neurogenic inflammation, bronchial asthma, arthritis, inflammatory bowel disease, gout, allergic and vasomotor rhinitis, eczema, urticaria or hives, and psoriasis.

5. The method of claim 2, wherein said pain is selected from the group consisting of nociceptive pain, neurogenic pain, postherpetic neuralgia, pain associated with diabetic neuropathy, pain associated with chronic peripheral polyneuropathy, stump pain after amputation, postmastectomy pain syndrome, pain associated with osteoarthritis, pain associated with Gillain-Barrés disease, headache, and itching.

6. The method of claim 5, wherein said headache is selected from the group consisting of migraine and Horton's headache.

7. The method of claim 3, wherein said allergy or autoimmune disease is selected from the group consisting of rheumatoid arthritis, conjunctivitis, rhinitis, and inflammatory bowel disease.

8. The method of claim 3, wherein said organ dysfunction is selected from the group consisting of osteoarthritis, nasopharyngeal adenoids, bronchial asthma, atherosclerosis, urge incontinence or bladder hyper-reactivity, cough, gastroduodenal ulcer or other mucosal damage in the gastrointestinal tract, emesis, myocardial infarction, unstable angina, septic shock, hemorrhagic shock, cardiac shock, cerebral vasospasm after subarachnoid hemorrhage, stroke, and benign and malignant tumors.

9. The method of claim 3, wherein said infection is selected from the group consisting of infection by a bacterium, infection by a virus, and infection by a parasite.

10. The method of claim 9, wherein said virus is a herpesvirus.

11. A method of achieving analgesia, said method comprising administering a compound that is structurally related to anandamide, AM404, 1-arachidonylglycerol, or 2-arachidonylglycerol to an individual in an amount sufficient to achieve analgesia,
wherein the compound that is structurally related to anandamide, AM404, 1-arachidonylglycerol, or 2-arachidonylglycerol can be represented by formula (I) or formula (II),
wherein formula (I) is:
A-B-C
in which A can be represented by 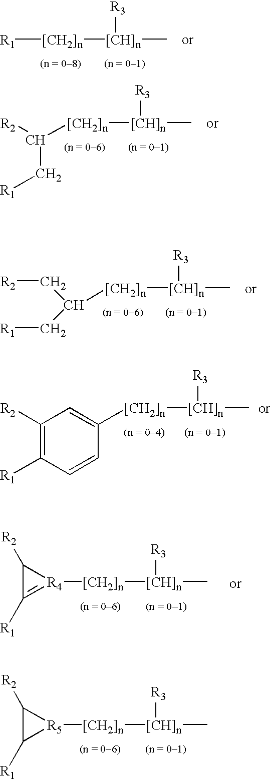
wherein R₁ can be any of the following substituents: —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br, and I; and
wherein R₂ can be any of the following substituents: —H, —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br and I;
wherein R₂ is not hydrogen when R₁ is alkoxy; and wherein any hydroxy group of R₁ and R₂ may be protected by a metabolically deprotectable protecting group to provide —OH in situ; and
wherein R₃ can be —H, —CH₃, —C₂H₅, and CF₃; and
wherein R₄ can be 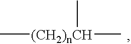
wherein n is 0-4; and
wherein R₅ can be 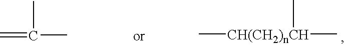
wherein n is 0-3; and
in which B can be represented by —NHC(O)—, —NHC(S)—, —NHC(O)NH—, —NHS(O)—, —C(O)O—, —C(O)S—, —C(S)O—, —NHS—, —C(O)NH—, —C(S)NH—, —NHC(S)NH—, —S(O)NH—, —OC(O)—, —SC(O)—, —OC(S)— or —SNH—; and
in which C can be represented by an unsaturated straight or branched hydrocarbon chain, containing 6 to 24 carbon atoms, preferably 12 to 22 carbon atoms, and at least one double bond; and
wherein formula (II) is:
D-E-C
in which D can be represented by 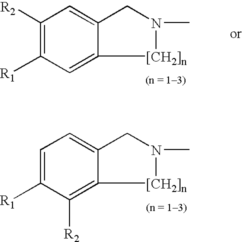
wherein R₁ can be any of the following, substituents: —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br, and I; and
wherein R₂ can be any of the following substituents: —H, —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br and I;
wherein R₂ is not hydrogen when R₁ is alkoxy; and wherein any hydroxy group of R₁ and R₂ may be protected by a metabolically deprotectable protecting group to provide —OH in situ; and
in which E can be represented by —C(O)—, —C(S)—, —C(O)NH—, —C(S)NH—, —S(O)—, —S—, —O—, —C(O)O—, —C(O)S—, —OC(O)—, or C(S)O—; and
in which C can be represented by an unsaturated straight or branched hydrocarbon chain, containing 6 to 24 carbon atoms, preferably 12 to 22 carbon atoms, and at least one double bond.

12. The method of claim 11, wherein said administering comprises contacting skin or a mucous membrane, or injection locally, epidurally, or spinally.

13. A method of developing agonists and antagonists of a vanilloid receptor, said method comprising
a) obtaining a compound that is structurally related to anandamide, AM404, 1-arachidonylglycerol, or 2-arachidonylglycerol,
wherein the compound that is structurally related to anandamide, AM404, 1-arachidonylglycerol, or 2-arachidonylglycerol can be represented by formula (I) or formula (TI),
wherein formula (I) is:
A-B-C
in which A can be represented by 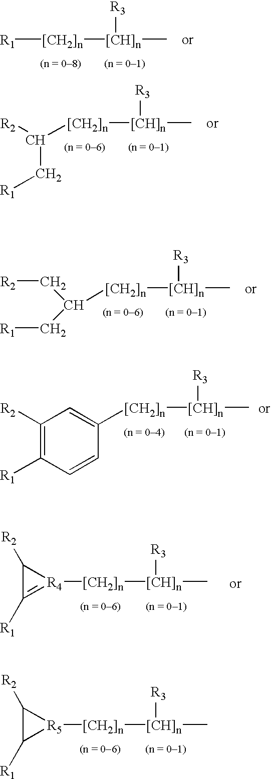
wherein R₁ can be any of the following substituents: —OH, —CH₂OH, —C₂H₅OH, —C₁₋₃alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br, and I; and
wherein R₂ can be any of the following substituents: —H, —OH, —CH₂OH, —C₂H₅OH, —C₁₋₃-alkoxy, —CH₂OCH₃, —C₂H₅CH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br and I;
wherein R₂ is not hydrogen when R₁ is alkoxy; and wherein any hydroxy group of R₁ and R₂ may be protected by a metabolically deprotectable protecting group to provide —OH in situ; and
wherein R₃ can be —H, —CH₃, —C₂H₅, and CF₃; and
wherein R₄ can be 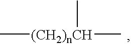
wherein n is 0-4; and
wherein R₅ can be 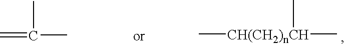
wherein n is 0-3; and
in which B can be represented by —NHC(O)—, —NHC(S)—, —NHC(O)NH—, —NHS(O)—, —C(O)O—, —C(O)S—, —C(S)O—, —NHS—, —C(O)NH—, —C(S)NH—, —NHC(S)NH—, —S(O)NH—, —OC(O)—, —SC(O)—, —OC(S)— or —SNH—; and
in which C can be represented by an unsaturated straight or branched hydrocarbon chain, containing 6 to 24 carbon atoms, preferably 12 to 22 carbon atoms, and at least one double bond; and
wherein formula (II) is:
D-E-C
in which D can be represented by 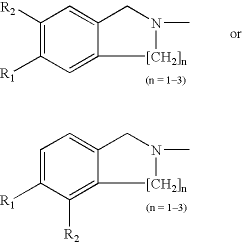
wherein R₁ can be any of the following substituents: —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br, and I; and
wherein R₂ can be any of the following substituents: —H, —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br and I;
wherein R₂ is not hydrogen when R₁ is alkoxy; and wherein any hydroxy group of R₁ and R₂ may be protected by a metabolically deprotectable protecting group to provide —OH in situ; and
in which E can be represented by —C(O)—, —C(S)—, —C(O)NH—, —C(S)NH—, —S(O)—, —S—, —O—, —C(O)O—, —C(O)S—, —OC(O)—, or C(S)O—; and
in which C can be represented by an unsaturated straight or branched hydrocarbon chain, containing 6 to 24 carbon atoms, preferably 12 to 22 carbon atoms, and at least one double bond; and
b) testing the compound for its ability to modulate the activity of at least one vanilloid receptor,
wherein modulation of activity indicates that the tested compound is an agonist or antagonist of a vanilloid receptor.

14. The method of claim 13, wherein said agonists and antagonists are obtained by chemical synthesis.

15. The method of claim 13, wherein said agonists and antagonists are obtained from biologically produced mixtures.

16. The method of claim 13, wherein said method is performed in vitro using cells expressing a recombinant VR1 receptor.

17. The method of claim 13, wherein said method is a high-throughput screening method.

18. A composition comprising a compound that is structurally related to anandamide, AM404, 1-arachidonylglycerol, or 2-arachidonylglycerol in an amount sufficient to modulate the in vivo activity of at least one vanilloid receptor,
wherein the compound that is structurally related to anandamide, AM404, 1-arachidonylglycerol, or 2-arachidonylglycerol can be represented by formula (1) or formula (II),
wherein formula (I) is:
A-B-C
in which A can be represented by 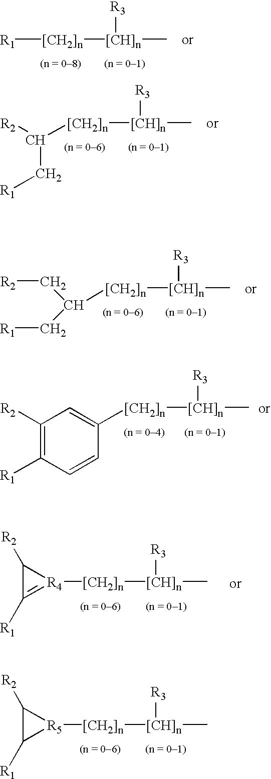
wherein R₁ can be any of the following substituents: —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br, and I; and
wherein R₂ can be any of the following substituents: —H, —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br and I;
wherein R₂ is not hydrogen when R₁ is alkoxy; and wherein any hydroxy group of R₁ and R₂ may be protected by a metabolically deprotectable protecting group to provide —OH in situ; and
wherein R₃ can be —H, —CH₃, —C₂H₅, and CF₃; and
wherein R₄ can be 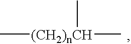
wherein n is 0-4; and
wherein R₅ can be 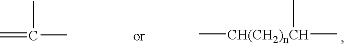
wherein n is 0-3; and
in which B can be represented by —NHC(O)—, —NHC(S)—, —NHC(O)NH—, —NHS(O)—, —C(O)O—, —C(O)S—, —C(S)O—, —NHS—, —C(O)NH—, —C(S)NH—, —NHC(S)NH—, —S(O)NH—, —OC(O)—, —SC(O)—, —OC(S)— or —SNH—; and
in which C can be represented by an unsaturated straight or branched hydrocarbon chain, containing 6 to 24 carbon atoms, preferably 12 to 22 carbon atoms, and at least one double bond; and
wherein formula (II) is:
D-E-C
in which D can be represented by 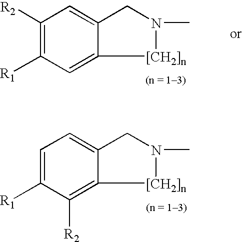
wherein R₁ can be any of the following substituents: —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br, and I; and
wherein R₂ can be any of the following substituents: —H, —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br and I;
wherein R₂ is not hydrogen when R₁ is alkoxy; and wherein any hydroxy group of R₁ and R₂ may be protected by a metabolically deprotectable protecting group to provide —OH in situ; and
in which E can be represented by —C(O)—, —C(S)—, —C(O)NH—, —C(S)NH—, —S(O)—, —S—, —O—, —C(O)O—, —C(O)S—, —OC(O)—, or C(S)O—; and
in which C can be represented by an unsaturated straight or branched hydrocarbon chain, containing 6 to 24 carbon atoms, preferably 12 to 22 carbon atoms, and at least one double bond.

19. The composition of claim 18, further comprising a drug.

20. A kit containing a compound that is structurally related to anandamide, AM404, 1-arachidonylglycerol, or 2-arachidonylglycerol in an amount sufficient to affect the in vivo activity of at least one vanilloid receptor,
wherein the compound that is structurally related to anandamide, AM404, 1-arachidonylglycerol, or 2-arachidonylglycerol can be represented by formula (I) or formula (II),
wherein formula (I) is:
A-B-C
in which A can be represented by 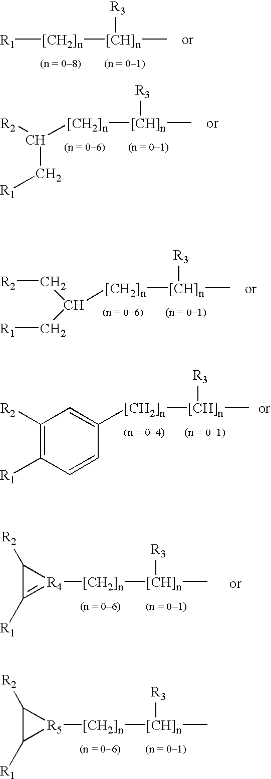
wherein R₁ can be any of the following substituents: —OH, —CH₂OH, —C₂H₅₀H, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br, and I; and
wherein R₂ can be any of the following substituents: —H, —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br and I;
wherein R₂ is not hydrogen when R₁ is alkoxy; and wherein any hydroxy group of R₁ and R₂ may be protected by a metabolically deprotectable protecting group to provide —OH in situ; and
wherein R₃ can be —H, —CH₃, —C₂H₅, and CF₃;
wherein R₄ can be 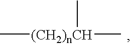
wherein n is 0-4; and
wherein R₅ can be 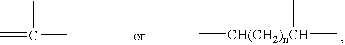
wherein n is 0-3; and
in which B can be represented by —NHC(O)—, —NHC(S)—, —NHC(O)NH—, —NHS(O)—, —C(O)O—, —C(O)S—, —C(S)O—, —NHS—, —C(O)NH—, —C(S)NH—, —NEC(S)NH—, —S(O)NH—, —OC(O)—, —SC(O)—, —OC(S)— or —SNH—; and
in which C can be represented by an unsaturated straight or branched hydrocarbon chain, containing 6 to 24 carbon atoms, preferably 12 to 22 carbon atoms, and at least one double bond; and
wherein formula (II) is:
D-E-C
in which D can be represented by 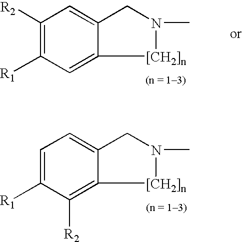
wherein R₁ can be any of the following substituents: —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H₅, —CH₂SCH₃, —C₂H₅SCH₃, —NO₂, —OCH₂NH₂, —OC₂H₅NH₂, Cl, F, Br, and I; and
wherein R₂ can be any of the following substituents: —H, —OH, —CH₂OH, —C₂H₅OH, —C₁₋₃-alkoxy, —CH₂OCH₃, —C₂H₅OCH₃, —OCH₂OH, —OC₂H₄OH, —OCH₂OCH₃, —OC₂H₄OCH₃, —SH, —CH₂SH, —C₂H₅SH, —SCH₃, —SC₂H, —CH₂SCH₃, —C₂H₅SCH₃, —NO₂, —OCH₂NH₂, —OC₂H₅NH₂, Cl, F, Br and I;
wherein R₂ is not hydrogen when R₁ is alkoxy; and wherein any hydroxy group of R₁ and R₂ may be protected by a metabolically deprotectable protecting group to provide —OH in situ; and
in which E can be represented by —C(O)—, —C(S)—, —C(O)NH—, —C(S)NH—, —S(O)—, —S—, —O—, —C(O)O—, —C(O)S—, —OC(O)—, or C(S)O—; and
in which C can be represented by an unsaturated straight or branched hydrocarbon chain, containing 6 to 24 carbon atoms, preferably 12 to 22 carbon atoms, and a least one double bond.

21. The kit of claim 20, wherein said kit further contains all the necessary compounds, solutions, and equipment for administration of anandamide, AM404, 1-arachidonylglycerol, or 2-arachidonylglycerol, or a structurally related lipid to an individual.
